(19) United States
(12) Patent Application Publication
(10) Pub. No.: US 2026/0275278 A1
(43) Pub. Date: Sep. 17, 2026

(54) METHOD OF GENERATING PANCREATIC BETA CELLS

(71) Applicant: Bit Bio Limited, Cambridge (GB)

(72) Inventors: Luca Crepaldi, Cambridge (GB); Elena Galimberti, Cambridge (GB); Sarah Gillen, Cambridge (GB); Emmanouil Metzakopian, Cambridge (GB); Anna Wilczynska, Cambridge (GB)

(21) Appl. No.: 19/668,205

(22) Filed: May 5, 2026

Related U.S. Application Data
(63) Continuation of application No. PCT/GB2024/052806, filed on Nov. 6, 2024.

(30) Foreign Application Priority Data
Nov. 6, 2023 (EP) 23208077.0
Jun. 25, 2024 (EP) 24386074.9

(51) Int. Cl. C12N 5/071; C07K 14/47

(52) U.S. Cl. CPC C12N 5/0613; C07K 14/4702; C12N 2501/91; C12N 2510/00

(57) ABSTRACT

The invention relates to methods of generating pancreatic beta cells using forward reprogramming.

### FIELD OF THE INVENTION

The invention relates to methods of generating pancreatic beta cells by overexpressing one or more polypeptides that have the activity of combinations of transcription factors and/or combinations of transcription factors themselves, i.e. through forward reprogramming or reprogramming.

### BACKGROUND OF THE INVENTION

Pancreatic beta cells—contained in clusters called islets—produce insulin and are critical in the management of blood glucose levels. Patients with diabetes (type 1 and type 2) have either dysfunctional or absent pancreatic beta cells. Current methods for management of type 1 diabetes generally require frequent glucose monitoring and life-long insulin administration. Transplantation of healthy islet and pancreatic beta cells to replace the lost or damaged cells has the potential to provide a breakthrough therapy for patients with diabetes. However, there is a shortage of donor organs which makes it difficult to source islet cells suitable for transplantation.

Currently, methods for differentiating stem cells into pancreatic beta cells generally include treating stem cells with a cocktail of differentiation-inducing materials and culturing them for a long time. However, these materials are expensive and the efficiency of cell differentiation is low. By contrast, reprogramming and forward programming strategies provide mature human cell types with unprecedented speed and efficiency (Zhang et al. (2013) Neuron 78:785-98). Forward programming involves directly converting pluripotent stem cells, including hPSCs, to mature cell types through the forced expression of polypeptides having the activity of key lineage transcription factors and/or the key lineage transcription factors themselves, in order to convert the stem cell into a particular mature cell type. Reprogramming is an alternative strategy whereby a somatic, differentiated cell, is converted into a particular, different mature cell type, again through the forced expression of polypeptides having the activity of key lineage transcription factors and/or the key lineage transcription factors themselves.

There a need in the art to provide methods for generating pancreatic beta cells suitable for use as potential therapeutic agents, in research and in tissue engineering.

### SUMMARY OF THE INVENTION

According to a first aspect of the invention, there is provided a method of generating pancreatic beta cells comprising expressing one or more polypeptides having the activity of one or more transcription factors and/or increasing the expression of one or more transcription factors, the transcription factors selected from the group consisting of: GLIS3, PDX1, NEUROD1, NKX6-2, ONECUT1 and variants thereof, in a cell population and culturing the cell population to obtain pancreatic beta cells.

According to a further aspect of the invention, there is provided a method of generating pancreatic beta cells comprising expressing one or more polypeptides having the activity of one or more transcription factors and/or increasing the expression of one or more transcription factors in a cell population and culturing the cell population to obtain pancreatic beta cells, the transcription factors comprising PDX1, GLIS3 and one or more additional transcription factors selected from the group consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1.

According to a further aspect of the invention, there is provided a method for the production of pancreatic beta cells from a source cell, comprising the steps of:
a) insertion of a gene encoding a transcriptional regulator protein into a first genomic safe harbour site of the source cell; and
b) insertion of at least one nucleotide sequence encoding one or more polypeptides having the activity of one or more transcription factors and/or encoding one or more transcription factors, the transcription factors selected from the group consisting of: GLIS3, PDX1, NEUROD1, NKX6-2, ONECUT1 and variants thereof, operably linked to an inducible promoter into one or more further genomic safe harbour sites of the source cell, wherein said inducible promoter is regulated by the transcriptional regulator protein; and
c) culturing the source cell(s) comprising the insertions to obtain pancreatic beta cells.

According to a further aspect of the invention, there is provided a method for the production of pancreatic beta cells from a source cell, comprising the steps of:
a) insertion of a gene encoding a transcriptional regulator protein into a first genomic safe harbour site of the source cell; and
b) insertion of at least one nucleotide sequence encoding one or more polypeptides having the activity of one or more transcription factors and/or encoding one or more transcription factors, the transcription factors comprising PDX1, GLIS3 and one or more additional transcription factors selected from the group consisting of: (1) NEUROD1, (2) ONECUT1, (3) MNX1, (4) FOXK1, (5) ASCL1, (6) NEUROD1 in combination with FOXK1, (7) NEUROD1 in combination with ONECUT1 and variants thereof, operably linked to an inducible promoter into one or more further genomic safe harbour sites of the source cell, wherein said inducible promoter is regulated by the transcriptional regulator protein; and
c) culturing the source cell(s) comprising the insertions to obtain pancreatic beta cells.

According to a further aspect of the invention, there is provided a method for the production of pancreatic beta cells from a source cell, comprising the steps of:
a) targeted insertion of a gene encoding a transcriptional regulator protein into a first genomic safe harbour site of the source cell; and
b) targeted insertion of at least one nucleotide sequence encoding one or more polypeptides having the activity of one or more transcription factors and/or encoding one or more transcription factors, the transcription factors selected from the group consisting of: GLIS3, PDX1, NEUROD1, NKX6-2, ONECUT1 and variants thereof, operably linked to an inducible promoter into one or more further genomic safe harbour sites of the source cell, wherein said inducible promoter is regulated by the transcriptional regulator protein; and
c) culturing the source cell(s) comprising the insertions to obtain pancreatic beta cells.

According to a further aspect of the invention, there is provided a use of one or more polypeptides having the activity of one or more transcription factors and/or one or more transcription factors, wherein the one or more transcription factors are selected from the group consisting of: GLIS3, PDX1, NEUROD1, NKX6-2, ONECUT1 and variants thereof, to generate pancreatic beta cells.

According to a further aspect of the invention, there is provided a use of one or more polypeptides having the activity of one or more transcription factors and/or one or more transcription factors, wherein the one or more transcription factors comprise PDX1, GLIS3 and one or more additional transcription factors selected from the group consisting of: (1) NEUROD1, (2) ONECUT1, (3) MNX1, (4) FOXK1, (5) ASCL1, (6) NEUROD1 in combination with FOXK1, (7) NEUROD1 in combination with ONECUT1 and variants thereof, to generate pancreatic beta cells.

According to a further aspect of the invention, there is provided a cell obtainable by any one of the methods defined herein.

According to a further aspect of the invention, there is provided a cell comprising one or more exogenous expression cassettes comprising nucleotide sequences encoding one or more polypeptides having the activity of one or more transcription factors and/or encoding one or more transcription factors, wherein the one or more transcription factors are selected from the group consisting of: GLIS3, PDX1, NEUROD1, NKX6-2, ONECUT1 and variants thereof.

According to a further aspect of the invention, there is provided a cell comprising one or more exogenous expression cassettes comprising nucleotide sequences encoding one or more polypeptides having the activity of one or more transcription factors and/or encoding one or more transcription factors, wherein the one or more transcription factors comprise PDX1, GLIS3 and one or more additional transcription factors selected from the group consisting of: (1) NEUROD1, (2) ONECUT1, (3) MNX1, (4) FOXK1, (5) ASCL1, (6) NEUROD1 in combination with FOXK1, (7) NEUROD1 in combination with ONECUT1 and variants thereof.

According to a further aspect of the invention, there is provided a cell as defined herein, for use in therapy, in vitro diagnostics or drug screening.

According to a further aspect of the invention, there is provided a kit for differentiating a cell into a pancreatic beta cell comprising:
(i) a source cell and an agent that activates or increases the expression or amount of one or more transcription factors; and/or
(ii) one or more expression cassette(s) comprising nucleotide sequences encoding one or more polypeptides having the activity of one or more transcription factors and/or encoding one or more transcription factors, wherein the one or more transcription factors are selected from the group consisting of: GLIS3, PDX1, NEUROD1, NKX6-2, ONECUT1 and variants thereof.

According to a further aspect of the invention, there is provided a kit for differentiating a cell into a pancreatic beta cell comprising:
(i) a source cell and an agent that activates or increases the expression or amount of one or more transcription factors; and/or
(ii) one or more expression cassette(s) comprising nucleotide sequences encoding one or more polypeptides having the activity of one or more transcription factors and/or encoding one or more transcription factors, wherein the one or more transcription factors comprise PDX1, GLIS3 and one or more additional transcription factors selected from the group consisting of: (1) NEUROD1, (2) ONECUT1, (3) MNX1, (4) FOXK1, (5) ASCL1, (6) NEUROD1 in combination with FOXK1, (7) NEUROD1 in combination with ONECUT1 and variants thereof.

According to a further aspect of the invention, there is provided a use of a kit as defined herein, for differentiating a cell into a pancreatic beta cell.

According to a further aspect of the invention, there is provided a method of drug screening comprising contacting a pancreatic beta cell generated using the method or a pancreatic beta cell as defined herein, with the drug and observing a change in the pancreatic beta cell induced by the drug.

According to a further aspect of the invention, there is provided a method of treating a subject having or at risk of a disease or disorder comprising administering to the subject a therapeutically effective amount of pancreatic beta cells generated using the method or pancreatic beta cells as defined herein.

In one embodiment, the transcription factors defined herein comprise PDX1 and GLIS3. In a further embodiment, the transcription factors defined herein comprise PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors defined herein comprise PDX1, GLIS3 and NEUROD1, preferably in combination with FOXK1 and/or ONECUT1.

### BRIEF DESCRIPTION OF THE FIGURES

FIG. 1. Quantitative PCR profiles of Insulin (upper panel) and MAFA (lower panel) gene expression in iPSCs reprogrammed with a pool of 5 eTFS, compared to all-minus-one pools or GLIS3 alone. Expression levels are relative to the housekeeping gene HMBS.

FIG. 2. Representative images of insulin C-peptide and MAFA expression as detected by immunofluorescence staining. Shown are expression profiles for iPSCs reprogrammed with a pool of 5 eTFS, compared to all-minus-one pools or GLIS3 alone. DAPI counterstaining is used to identify cell nuclei. Scale bar=130 μm.

FIG. 3. Single-cell gene expression data visualized on a UMAP plot. A) Highlighted are the cells for each replicate. B) Distribution of detected NEG1 control within the UMAP plot. C) Distribution of detected transcription factor GLIS3 within the UMAP plot. D) Distribution of detected insulin (INS) within the UMAP plot.

FIG. 4. Percentage of cells expressing each eTF (irrespective of the combination) in cells that also express Insulin. Cells that do not express any eTF are excluded from this analysis. The number above each set of bars indicates the ratio between the positive and negative percentages.

FIG. 5. Immunocytochemistry for the TF PDX1 and the pancreatic beta cells marker insulin C-peptide in a tetracycline-inducible system with different TF combinations on day 8 of reprogramming. Scale bar is 130 μm. PG: PDX1 and GLIS3; POG: PDX1, ONECUT1 and GLIS3; PNG: PDX1, NEUROD1 and GLIS3.

FIG. 6. RT-qPCR of beta cells genes in a tetracycline-inducible system with different TF combinations on day 8 of reprogramming.

### DETAILED DESCRIPTION

The present invention provides methods for producing pancreatic beta cells from source cells by expressing of one or more polypeptides having the activity of one or more transcription factors and/or increasing the expression of a select group of transcription factors which the present inventors have identified as inducing cell reprogramming into pancreatic beta cells.

### Definitions

Unless defined otherwise, all technical and scientific terms used herein have the meaning commonly understood by a person skilled in the art to which this invention belongs. As used herein, the following terms have the meanings ascribed to them below.

References to “transcription factor” as used herein, refer to proteins that are involved in gene regulation in both prokaryotic and eukaryotic organisms. In one embodiment, transcription factors can have a positive effect on gene expression and, thus, may be referred to as an “activator” or a “transcriptional activation factor”. In another embodiment, a transcription factor can negatively affect gene expression and, thus, may be referred to as “repressors” or a “transcription repression factor”. Activators and repressors are generally used terms and their functions may be discerned by those skilled in the art.

The term “increasing the expression of” or “increasing the amount of” with respect to increasing an amount, level or expression of a transcription factor, refers to increasing the quantity of the transcription factor in a cell of interest (e.g., a source cell). In some embodiments, the amount of transcription factor is increased in a cell (e.g., via an expression cassette directing expression of a polynucleotide encoding one or more transcription factors) when the quantity of transcription factor is at least 10%, 20%, 30%, 40%, 50%, 60%, 70%, 80%, 90%, or more relative to a control (e.g., a source cell without said expression cassette(s) or a control cell where the baseline expression is zero or negligible). In some of the embodiments, increasing the expression comprises “overexpressing” the transcription factor, i.e., increasing the expression of the transcription factor above the endogenous expression level of the transcription factor in the cell.

Methods of the invention may be used in a “cell population”, i.e., a collection of cells which may be differentiated into the desired cell type. Said cell population may comprise “source cells”, also referred to as “starting cells”, i.e., a cell type prior to differentiation into the desired cell type.

References herein to “pluripotent” refer to cells which have the potential to differentiate into all types of cell found in an organism. One form of pluripotent stem cell, known as induced pluripotent stem cells, are of particular interest to the present invention. “Induced pluripotent stem cells” (iPSCs) are cells that have been reprogrammed to an embryonic stem cell-like state by being forced to express genes and factors important for maintaining the defining properties of embryonic stem cells. In 2006, it was shown that overexpression of four specific transcription factors could convert adult cells into pluripotent stem cells. OCT-3/4 and certain members of the SOX gene family have been identified as potentially crucial transcriptional regulators involved in the induction process. Additional genes including certain members of the KLF family, the MYC family, NANOG, and LIN28, may increase the induction efficiency. Examples of the genes which may be used as reprogramming factors to generate iPSCs include OCT3/4, SOX2, SOX1, SOX3, SOX15, SOX17, KLF4, KLF2, C-MYC, N-MYC, L-MYC, NANOG, LIN28, FOX15, ERAS, ECAT15-2, TCL1, CTNNB1, LIN28B, SALLI4, ESRRB, TBX3 and GLIS1, GATA3, GATA6 and these reprogramming factors may be used singly, or in combination of two or more kinds thereof. In particular, the reprogramming factors may comprise at least the Yamanaka factors, i.e., OCT3/4, SOX2, KLF4 and C-MYC. These reprogramming factors may also be used in combination with the transcription factors of interest in the present invention.

References herein to “somatic” refer to any type of cell that makes up the body of an organism, excluding germ cells. Somatic cells therefore include, for example, skin, heart, muscle, bone or blood cells and their stem cells. Somatic cells may also be referred to as differentiated cells. In one embodiment, the somatic cell may be an adult cell or a cell derived from an adult which displays one or more detectable characteristics of an adult or non-embryonic cell.

Methods of the invention (e.g., cellular reprogramming of iPSCs) are for use in generating “pancreatic beta cells”, which may also be simply referred to as “beta cells”. The term “pancreatic beta cells” as used herein is meant to refer to cells that are found in pancreatic islets that synthesize and secrete insulin and amylin. The term “pancreatic beta cells” includes pancreatic beta-like cells that exhibit some but not all characteristics of adult pancreatic beta cells, as well as mature, fully functional and/or metabolically active adult pancreatic beta cells. This term includes further cells with the capacity to engraft pancreatic islets when transplanted in vivo. The pancreatic beta cells produced by this method may be at least as functional as the pancreatic beta cells produced by directed differentiation to date.

References herein to “culturing” include the addition of cells (e.g., the cell population, i.e., the source cells), to media comprising growth factors and/or essential nutrients. It will be appreciated that such culture conditions may be adapted according to the cells or cell population to be generated according to methods of the invention.

References to a “variant” when referring to a polypeptide could be, for example, an amino acid sequence at least 80%, 85%, 90%, 95%, 98%, or 99% identical to the original, full-length polypeptide. When referring to a nucleic acid sequence, the term “variant” could be, for example, a nucleic acid sequence at least 80%, 85%, 90%, 95%, 98%, or 99% identical to the original, full-length nucleic acid sequence. The variant could be a fragment of full-length polypeptide, in particular a functional fragment of the polypeptide. The fragment may be at least 50%, 60%, 70%, 80%, 85%, 90%, 95%, 98%, or 99% as long as the full-length wild type polypeptide or a domain thereof having an activity of interest such as the ability to differentiate a source cell into a pancreatic beta cell. Variations known in the art to eliminate or substantially reduce the activity of the protein are preferably avoided. In some embodiments, the variant lacks an N- and/or C-terminal portion of the full-length polypeptide, e.g., up to 10, 20, or 50 amino acids from either terminus is lacking. In some embodiments, a functional variant or fragment has at least 50%, 60%, 70%, 80%, 90%, 95% or more of the activity of the full-length wild type polypeptide. One of skill in the art will be aware of, or will readily be able to ascertain, whether a particular polypeptide variant or fragment is functional using assays known in the art. For example, the ability of a variant of a transcription factor as listed in Table 1 to generate pancreatic beta cells can be assessed using the assays as described herein. In particular, the variant may be a biologically active variant. A “biologically active variant” includes any variant of a molecule having substantially, at least in part, the same functional and/or biological properties of said molecule, such as binding properties, and/or the same structural features, such as binding domain. It also refers to a molecule that exhibits the functional features as the transcription factors disclosed herein.

In one embodiment, the variant is an isoform of the listed transcription factor. Many transcription factors have one or more isoforms which result, for example, from alternative splicing or from a shifted transcription initiation. Based on the different transcript variants (i.e. mRNA), different polypeptides are generated. It is possible that different transcript variants have different translation initiation sites.

A “promoter” is a nucleotide sequence which is recognised by proteins involved in initiating and regulating transcription of a polynucleotide sequence. An “inducible promoter” is a nucleotide sequence where expression of a genetic sequence operably linked to the promoter is controlled by an analyte, co-factor, regulatory protein, etc. It is intended that the term “promoter” or “control element” includes full-length promoter regions and functional (e.g., controls and/or affects transcription or translation) segments of these regions.

The term “operably linked” refers to an arrangement of elements wherein the components so described are configured so as to perform their usual function. Thus, a given promoter operably linked to a genetic sequence is capable of effecting the expression of that sequence when the regulatory factors are present. The promoter need not be contiguous with the sequence, so long as it functions to direct the expression thereof. Thus, for example, intervening untranslated yet transcribed sequences can be present between the promoter sequence and the genetic sequence and the promoter sequence can still be considered “operably linked” to the genetic sequence. Thus, the term “operably linked” is intended to encompass any spacing or orientation of the promoter element and the genetic sequence in the inducible cassette which allows for initiation of transcription of the inducible cassette upon recognition of the promoter element by a transcription complex.

The term “vector”, as used herein, is intended to refer to a nucleic acid molecule which is used as a vehicle to carry genetic material into a cell. One type of vector is a “plasmid”, which refers to a circular double stranded DNA loop or circle into which additional DNA segments may be ligated. Another type of vector is an infectious but non-pathogenic viral vector, wherein additional DNA segments may be ligated to certain viral genetic elements. Certain vectors are capable of autonomous replication in a host cell into which they are introduced (e.g., bacterial vectors having a bacterial origin of replication and episomal mammalian and yeast vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. However, the invention is intended to include such other forms of expression vectors, such as viral vectors (e.g., replication defective retroviruses, lentiviral vectors, adenoviruses, Sendai viruses and adeno-associated viruses), which serve equivalent functions, and also bacteriophage and phagemid systems. Another type of vector includes synthetic and in vitro transcribed RNA molecules, e.g., mRNA and stabilised RNA, to carry coding genetic information to the cells. This also includes synthetic-self-replicating RNA vectors.

References to “subject”, “patient” or “individual” refer to a subject, in particular a mammalian subject, to be treated. Mammalian subjects include humans, non-human primates, farm animals (such as cows), sports animals, or pet animals, such as dogs, cats, guinea pigs, rabbits, rats or mice. In some embodiments, the subject is a human. In alternative embodiments, the subject is a non-human mammal, such as a mouse.

The term “sufficient amount” means an amount sufficient to produce a desired effect. The term “therapeutically effective amount” is an amount that is effective to ameliorate a symptom of a disease or disorder. A therapeutically effective amount can be a “prophylactically effective amount” as prophylaxis can be considered therapy.

As used herein, the term “about” when used herein includes up to and including 10% greater and up to and including 10% lower than the value specified, suitably up to and including 5% greater and up to and including 5% lower than the value specified, especially the value specified. The term “between” includes the values of the specified boundaries.

It will be understood that any method as described herein may have one or more, or all, steps performed in vitro, ex vivo or in vivo.

### Transcription Factor Activity

The method described herein may comprise increasing the expression (in particular, the protein expression) of a sufficient number of polypeptides having the activity of the transcription factors (or the transcription factors themselves) (e.g., as listed in Table 1 and variants and isoforms thereof) capable of causing differentiation of a cell population to pancreatic beta cells, therefore differentiating the cell population into pancreatic beta cells. In the context of the present invention, these factors may also be referred to as “reprogramming factors”. As described herein, the expression of an exogenous or endogenous (in particular an exogenous) transcription factor may be increased.

According to an aspect of the invention, there is provided a method of generating pancreatic beta cells comprising expressing one or more polypeptides having the activity of one or more transcription factors and/or increasing the expression of one or more transcription factors selected from the group consisting of: GLIS3, PDX1, NEUROD1, NKX6-2, ONECUT1 and variants thereof, in a cell population and culturing the cell population to obtain pancreatic beta cells.

References herein to “GLIS3” relates to GLIS Family Zinc Finger 3. In one embodiment, the GLIS3 is human GLIS3. Wild type human GLIS3 is identified by UniProt ID: Q8NEA6, and is encoded by the GLIS3 gene, identified by Ensembl Gene ID: ENSG00000107249. GLIS3 functions as a bifunctional mediator of the Sonic Hedgehog (SHH) pathway, providing activating and repressing transcription functions.

References herein to “PDX1” relates to Pancreatic and Duodenal homeobox 1 (also known as insulin promoter factor 1). In one embodiment, the PDX1 is human PDX1. Wild type human PDX1 is identified by UniProt ID: P52945, and is encoded by the PDX1 gene, identified by Ensembl Gene ID: ENSG00000139515. PDX1 is required for the formation of the endocrine pancreas. PDX1 targets PDX1 itself (as a form of auto-regulation), regulatory factor X 6 (RFX6), hepatocyte nuclear factor 1 homeobox B (HNF1B) and Meis homeobox 1 (MEIS1).

References herein to “NEUROD1” relates to Neurogenic differentiation 1 (also known as β2). In one embodiment, the NEUROD1 is human NEUROD1. Wild type human NEUROD1 is identified by UniProt ID: Q13562, and is encoded by the NEUROD1 gene, identified by Ensembl Gene ID: ENSG00000162992. NEUROD1 is a basic helix-loop-helix (bHLH) transcription factor that forms heterodimers with other bHLH proteins and activates the transcription of genes containing an E-box DNA sequence.

References herein to “NKX6-2” relates to Homeobox protein Nkx-6.2 (also known as NK6 homeobox 2). In one embodiment, the NKX6-2 is human NKX6-2. Wild type human NKX6-2 is identified by UniProt ID: Q9C056, and is encoded by the NKX6-2 gene, identified by Ensembl Gene ID: ENSG00000148826. NKX6-2 is transcriptional repressor of factors such as developing brain homeobox protein 1 (DBX1).

References herein to “ONECUT1” relates to One cut homeobox 1 (also known as Hepatocyte nuclear factor 6 or HNF6). In one embodiment, the ONECUT1 is human ONECUT1. Wild type human ONECUT1 is identified by UniProt ID: Q9UBC0, and is encoded by the ONECUT1 gene, identified by Ensembl Gene ID: ENSG00000169856. ONECUT1 controls the diversification of motor neurons by ensuring the maintenance of Isl1 expression during differentiation.

In one embodiment, the method comprises expressing one or more polypeptides having the activity of two or more transcription factors, in particular three or more, four or more or five transcription factors and/or increasing the expression of two or more transcription factors, in particular three or more, four or more or five transcription factors, the transcription factors selected from the group consisting of: GLIS3, PDX1, NEUROD1, NKX6-2, ONECUT1; and variants thereof. Preferably the transcription factors comprise or consist of PDX1 and GLIS3. More preferably the transcription factors comprise or consist of PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors defined herein comprise or consist of PDX1, GLIS3 and NEUROD1, preferably in combination with FOXK1 and/or ONECUT1. In a further embodiment, the transcription factors defined herein comprise or consist of PDX1, GLIS3 and FOXK1, preferably in combination with NEUROD1 and/or ONECUT1. In a further embodiment, the transcription factors defined herein comprise or consist of PDX1, GLIS3 and ONECUT1, preferably in combination with NEUROD1 and/or FOXK1. In one embodiment the transcription factors comprise or consist of PDX1, GLIS3 and ONECUT1. In one embodiment the transcription factors comprise or consist of PDX1, GLIS3 and MNX1. In one embodiment the transcription factors comprise or consist of PDX1, GLIS3 and FOXK1. In one embodiment the transcription factors comprise or consist of PDX1, GLIS3 and ASCL1. In one embodiment the transcription factors comprise or consist of PDX1, GLIS3, NEUROD1 and FOXK1. In one embodiment the transcription factors comprise or consist of PDX1, GLIS3, NEUROD1 and ONECUT1. In one embodiment the transcription factors comprise or consist of PDX1, GLIS3, NEUROD1, FOXK1 and ONECUT1.

References herein to “MNX1” relates to Motor neuron and pancreas homeobox 1 (also known as Homeobox HB9 (HLXB9)). In one embodiment, the MNX1 is human MNX1. Wild type human MNX1 is identified by UniProt ID: P50219, and is encoded by the MNX1 gene, identified by Ensembl Gene ID: ENSG00000130675. MNX1 negatively modulates visual system homeobox CHX10 and blocks regulatory sequence interactions of the ISL1-LHX3 complex.

References herein to “FOXK1” relates to Forkhead box K1. In one embodiment, the FOXK1 is human FOXK1. Wild type human FOXK1 is identified by UniProt ID: P85037, and is encoded by the FOXK1 gene, identified by Ensembl Gene ID: ENSG00000164916. FOXK1 binds to the forkhead DNA sequence 5′-GTAAACA-3′. FOXK1 promotes expression of enzymes for glycolysis (such as hexokinase-2 (HK2), phosphofructokinase, pyruvate kinase (PKLR) and lactate dehydrogenase), while suppressing further oxidation of pyruvate in the mitochondria by up-regulating pyruvate dehydrogenase kinases PDK1 and PDK4.

References herein to “ASCL1” relates to Achaete-scute homolog 1. In one embodiment, the ASCL1 is human ASCL1. Wild type human ASCL1 is identified by UniProt ID: P50553, and is encoded by the ASCL1 gene, identified by Ensembl Gene ID: ENSG00000139352. ASCL1 directly binds to the E box motif (5′-CANNTG-3′) on promoters and promotes transcription of neuronal genes.

Once the activity of a transcription factor is appreciated, the endogenous transcription machinery can be modulated using not only the transcription factors themselves, but also polypeptides engineered to replicate the action of the transcription factor, such as synthetic transcription factors or artificial transcription factors. For example, CRISPR (clustered regularly interspaced palindromic repeats), TALE (transcriptional activator-like effector) or Zinc Finger technologies can be used to modulate the expression of endogenous cellular genes, to allow for faster and more efficient nuclear reprogramming under conditions amenable for clinical and commercial applications. This is set out in, for example, US2016/362705, incorporated herein.

Alternatively, with the development of highly accurate protein structure prediction with artificial intelligence tools such as AlphaFold, it is now straightforward for polypeptides to be developed that have very similar structure and/or activity to a transcription factor of interest whilst at the same time having an amino acid sequence that has very little resemblance to that of the transcription factor of interest. For example, large language models trained on biological diversity have been used to develop proteins only around 70% identical to CRISPR-Cas proteins that occur in nature and yet with comparable or improved biological activity and specificity (Ruffolo et al. (2024) bioRxiv, doi: https://doi.org/10.1101/2024.04.22.590591). Such polypeptides are covered within the scope of the invention.

In some embodiments of the present invention, a polypeptide (in particular a single polypeptide) is engineered to mimic the activity of more than one transcription factor of interest. In a further embodiment, polypeptides having the activity of one or more transcription factors is expressed, in combination with increasing the expression of another transcription factor. For example, a polypeptide having the activity of PDX1 can be expressed in combination with increasing the expression of GLIS3 (or vice versa).

According to another aspect of the invention, there is provided a method of generating pancreatic beta cells comprising increasing the activity of one or more transcription factors, in a non-beta cell population (e.g. iPSCs) and culturing the cell population to obtain pancreatic beta cells, wherein the transcription factors are selected from the group consisting of: GLIS3, PDX1, NEUROD1, NKX6-2 and ONECUT1. Activity of the transcription factors may be increased, for example, by introducing the transcription factor itself or introducing polypeptides which increase the expression of the transcription factor. In one embodiment, activity of GLIS3, PDX1, NEUROD1, NKX6-2 or ONECUT1 is increased by expressing one or more polypeptides having the activity of GLIS3, PDX1, NEUROD1, NKX6-2 or ONECUT1 and/or increasing the expression of GLIS3, PDX1, NEUROD1, NKX6-2 or ONECUT1.

According to another aspect of the invention, there is provided a method of generating pancreatic beta cells comprising expressing one or more polypeptides having the activity of one or more transcription factors, and/or increasing the expression of one or more transcription factors, wherein the one or more transcription factors are selected from the group consisting of: GLIS3, PDX1, NEUROD1, NKX6-2, ONECUT1; and variants thereof, in a non-pancreatic beta cell population (e.g. iPSCs) and culturing the cell population to obtain pancreatic beta cells.

In one embodiment, the transcription factors comprise PDX1 and GLIS3. In a further embodiment, the transcription factors comprise PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors comprise PDX1, GLIS3 and NEUROD1, preferably in combination with FOXK1 and/or ONECUT1.

In one embodiment, the (e.g. one or more) transcription factors are selected from the group consisting of: GLIS3, PDX1, NEUROD1, NKX6-2, ONECUT1 and variants thereof.

In a further embodiment, the method comprises increasing the expression of GLIS3 and PDX1. In a yet further embodiment, the method comprises increasing the expression of GLIS3, PDX1 and NEUROD1.

In one embodiment, the method comprises expressing one or more polypeptides having the activity of one or more additional transcription factors, and/or increasing the expression of one or more additional transcription factors. The additional transcription factors may be one or more of the transcription factors listed in Table 1.

In one embodiment, the transcription factor comprises GLIS3. GLIS3 may be used in combination with one or more, such as one, two, three, four, or five transcription factors selected from the list in Table 1. In a further embodiment, the method comprises expressing one or more polypeptides having the activity of between two and five transcription factors, and/or increasing the expression of between two and five transcription factors, the transcription factors selected from GLIS3 in combination with PDX1, NEUROD1, NKX6-2, ONECUT1, or variants thereof.

In one embodiment, the transcription factor comprises PDX1. PDX1 may be used in combination with one or more, such as one, two, three, four, or five transcription factors selected from the list in Table 1. In a further embodiment, the method comprises expressing one or more polypeptides having the activity of between two and five transcription factors, and/or increasing the expression of between two and five transcription factors, the transcription factors selected from PDX1 in combination with GLIS3, NEUROD1, NKX6-2, ONECUT1, or variants thereof.

In one embodiment, the transcription factor comprises NEUROD1. NEUROD1 may be used in combination with one or more, such as one, two, three, four, or five transcription factors selected from the list in Table 1. In a further embodiment, the method comprises expressing one or more polypeptides having the activity of between two and five transcription factors, and/or increasing the expression of between two and five transcription factors, the transcription factors selected from NEUROD1 in combination with GLIS3, PDX1, NKX6-2, ONECUT1, or variants thereof.

In one embodiment, the transcription factor comprises NKX6-2. NKX6-2 may be used in combination with one or more, such as one, two, three, four, or five transcription factors selected from the list in Table 1. In a further embodiment, the method comprises expressing one or more polypeptides having the activity of between two and five transcription factors, and/or increasing the expression of between two and five transcription factors, the transcription factors selected from NKX6-2 in combination with GLIS3, PDX1, NEUROD1, ONECUT1, or variants thereof.

In one embodiment, the transcription factor comprises ONECUT1. ONECUT1 may be used in combination with one or more, such as one, two, three, four, or five transcription factors selected from the list in Table 1. In a further embodiment, the method comprises expressing one or more polypeptides having the activity of between two and five transcription factors, and/or increasing the expression of between two and five transcription factors, the transcription factors selected from ONECUT1 in combination with GLIS3, PDX1, NEUROD1, NKX6-2, or variants thereof.

Methods of the invention encompass the use of variants of the transcription factors of interest (i.e., as described in Table 1). References to the transcription factors also encompasses species variants, isoforms, homologues, allelic forms, mutant forms, and equivalents thereof, including conservative substitutions, additions, deletions therein not adversely affecting the structure and/or function. Changes in the nucleic acid sequence of the transcription factor gene can result in conservative changes or substitutions in the amino acid sequence. Therefore, the invention includes polypeptides having conservative changes or substitutions. The invention includes sequences where conservative substitutions are made that do not alter the activity of the transcription factor protein of interest.

References to a “variant” when referring to a polypeptide could be an amino acid sequence at least 80%, 85%, 90%, 95%, 98%, or 99% identical to the original, full-length polypeptide. When referring to a nucleic acid sequence, the term “variant” could be, for example, a nucleic acid sequence at least 80%, 85%, 90%, 95%, 98%, or 99% identical to the original, full-length nucleic acid sequence. The variant could be a fragment of full-length polypeptide, in particular a functional fragment of the polypeptide. The fragment may be at least 50%, 60%, 70%, 80%, 85%, 90%, 95%, 98%, or 99% as long as the full-length wild type polypeptide or a domain thereof having an activity of interest such as the ability to forward-program a source cell into a microglial cell. Variations known in the art to eliminate or substantially reduce the activity of the protein are preferably avoided. In some embodiments, the variant lacks an N- and/or C-terminal portion of the full-length polypeptide, e.g., up to 10, 20, or 50 amino acids from either terminus is lacking. In some embodiments, a functional variant or fragment has at least 50%, 60%, 70%, 80%, 90%, 95% or more of the activity of the full-length wild type polypeptide. One of skill in the art will be aware of, or will readily be able to ascertain, whether a particular polypeptide variant or fragment is functional using assays known in the art. For example, the ability of a variant of a transcription factor as listed in Table 1 to generate pancreatic beta cells can be assessed using the assays as described herein. In particular, the variant may be a biologically active variant. A “biologically active variant” includes any variant of a molecule having substantially, at least in part, the same functional and/or biological properties of said molecule, such as binding properties, and/or the same structural features, such as binding domain. It also refers to a molecule that exhibits the same functional features as the transcription factors disclosed herein.

In one embodiment, an isoform of the listed transcription factor is used. Many transcription factors have one or more isoforms which result, for example, from alternative splicing or from a shifted transcription initiation. Based on the different transcript variants (i.e. mRNA), different polypeptides are generated. It is possible that different transcript variants have different translation initiation sites.

TABLE 1
Transcription factors for generation of pancreatic beta cells,
including accession numbers (as accessed on 6 Nov. 2023)

| | Transcription Factor | |
|---|---|---|
| Transcription Factor Protein name | Gene name | Ensembl Gene ID |
| GLIS Family Zinc Finger 3 | GLIS3 | ENSG00000107249 |
| Pancreatic and duodenal homeobox 1 | PDX1 | ENSG00000139515 |
| Neurogenic differentiation 1 | NEUROD1 | ENSG00000162992 |
| Homeobox protein Nkx-6.2 | NKX6-2 | ENSG00000148826 |
| One cut homeobox 1 | ONECUT1 | ENSG00000169856 |
| Basic helix-loop-helix family member e41 | BHLHE41 | ENSG00000123095 |
| TEA domain transcription factor 1 | TEAD1 | ENSG00000187079 |
| Regulatory factor X2 | RFX2 | ENSG00000087903 |
| CCCTC-binding factor like | CTCFL | ENSG00000124092 |
| Myelin transcription factor 1 like | MYT1L | ENSG00000186487 |
| Transcription factor Maf | MAF | ENSG00000178573 |
| Retinoic acid receptor alpha | RARA | ENSG00000131759 |
| Basic helix-loop-helix family member e40 | BHLHE40 | ENSG00000134107 |
| Transcription factor MafB | MAFB | ENSG00000204103 |
| Achaete-scute homolog 1 | ASCL1 | ENSG00000139352 |
| Basic helix-loop-helix family member a15 | BHLHA15 | ENSG00000180535 |
| Forkhead box K1 | FOXK1 | ENSG00000164916 |
| Motor neuron and pancreas homeobox 1 | MNX1 | ENSG00000130675 |
| General transcription factor II-I repeat | GTF2IRD1 | ENSG00000006704 |
| domain-containing protein 1 | | |
| Insulin gene enhancer protein ISL-1 | ISL1 | ENSG00000016082 |
| Neurogenin 3 | NEUROG3 | ENSG00000122859 |

### Cell Types

The method may be used on any cell type, including stem cells. In the case of stem cells, the generation of pancreatic beta cells using the method may be referred to as “cellular reprogramming”, “forward reprogramming”, “direct programming” or “direct differentiation”, i.e., the pluripotent stem cell is differentiated into a pancreatic beta cell. Furthermore, pancreatic beta cellular reprogramming may be used as generic terminology referring to the use of transcription factors to differentiate a source cell into pancreatic beta cells.

Sources of cells suitable for methods of the invention may include, for example, any stem cells or non-pancreatic beta cells. For example, the stem cells may be pluripotent stem cells, for example induced pluripotent stem cells, embryonic stem cells or pluripotent stem cells derived by nuclear transfer or cell fusion. It may be preferred that the embryonic stem cell is derived without destruction of the embryo, particularly where the cells are human. In some embodiments, the stem cells are not derived from human or animal embryos, i.e., the invention does not extend to any methods which involve the destruction of human or animal embryos. The stem cells may also include multipotent stem cells, oligopotent stem cells, or unipotent stem cells. The stem cells may also include fetal stem cells or adult stem cells, such as hematopoietic stem cells, mesenchymal stem cells, neural stem cells, epithelial stem cells, skin stem cells. In certain aspects, the stem cells may be isolated from umbilical, placenta, amniotic fluid, chorionic villi, blastocysts, bone marrow, adipose tissue, brain, peripheral blood, cord blood, menstrual blood, blood vessels, skeletal muscle, skin and liver.

In one embodiment, the cell population is of human origin. The source cell e.g., a non-pancreatic beta cell, may be of human origin.

In one embodiment, the cell population is of animal origin. The source cell e.g., a non-pancreatic beta cell, may be of animal origin. In certain aspects, the cell is preferably one from a livestock animal. Livestock animals include, for example, pigs, cows, horses, buffalo, bison, goats, sheep, deer, reindeer, donkeys, bantengs, yaks, chickens, ducks and turkeys.

In one embodiment, the cell population comprises stem cells, e.g., induced pluripotent stem cells (iPSCs), embryonic stem cells (ESCs), haematopoietic stem cells, mesenchymal stem cells or neuronal stem cells. In a further embodiment, the cell population comprises pluripotent stem cells, e.g., iPSCs or ESCs.

In one embodiment, the source cell is a stem cell, e.g., an iPSC, an ESC, a haematopoietic stem cell, a mesenchymal stem cell or a neuronal stem cell. In a further embodiment, the source cell is a pluripotent stem cell, e.g., an iPSC or an ESC. In some embodiments, the source cell is an iPSC.

Methods of preparing induced pluripotent stem cells are also known in the art. Induction of iPSCs typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc. In one method, iPSC may be generated by transfecting cells with transcription factors Oct4, Sox2, c-Myc and Klf4 using viral transduction. In an alternative method, iPSC may be generated by transfecting cells with RNA encoding transcription factors inducing the development of stem cell characteristics, such as transcription factors selected from Oct4, Sox2, c-Myc and Klf4.

In one embodiment, the cell population comprises somatic cells, e.g., differentiated cells such as fibroblasts. The source cell may therefore be a somatic cell and the method may be a transdifferentiation method (i.e. the conversion of one cell type to another). Alternatively, the method may be used to convert a related somatic cell (e.g. pancreatic progenitor or endocrine progenitor cells) to a pancreatic beta cell.

In one embodiment, the pancreatic beta cells are human pancreatic beta cells.

In one embodiment, the induced pluripotent stem cells are derived from somatic or germ cells of the patient. Such use of autologous cells would remove the need for matching cells to a recipient. Alternatively, commercially available iPSC may be used, such as those available from WICELL (WiCell Research Institute, Inc, Wisconsin, US). Alternatively, the cells may be a tissue-specific stem cell which may also be autologous or donated.

### Delivery of Transcription Factors

It will be understood that methods for expressing polypeptides having transcription factor activity and/or increasing the expression of the transcription factors in the cells to be programmed into pancreatic beta cells may include any method known in the art, for example, by induction of expression of one or more expression cassettes previously introduced into the cells, or by introduction of nucleic acids (such as DNA or RNA), polypeptides, or small molecules to the cells to stimulate expression of the endogenous or exogenous transcription factors. Increasing the expression of certain endogenous but transcriptionally repressed genes may also reverse the silencing or inhibitory effect on the expression of these genes by regulating the upstream transcription factor expression or epigenetic modulation. Therefore, methods of the invention may involve culturing the cell population under conditions to artificially increase the expression level of one or more of the transcription factors described herein.

In one embodiment, the expression of the polypeptides having transcription factor activity and/or the transcription factors is increased by contacting the cell population with the polypeptides and/or the transcription factors (i.e., the proteins encoding the transcription factors). Delivery of the transcription factors may occur using direct electroporation of transcription factor proteins to the cells.

In an alternative embodiment, the expression of the transcription factors is increased by introducing a promoter (e.g. a strong promoter) ahead of an endogenous gene encoding the transcription factor(s).

In a further alternative embodiment, the expression of the polypeptides having transcription factor activity and/or the transcription factors is increased by contacting the cell population with one or more agents that activate or increase the expression amount of the (exogenous or endogenous) transcription factors. In the case of polypeptides having transcription factor activity or exogenous transcription factors, the agents may still be used after the genes for the polypeptides and/or transcription factors have been inserted into the cell.

In one embodiment, the agent is selected from the group consisting of: a nucleic acid (i.e., polynucleotide, e.g., messenger RNA (mRNA), coding DNA sequence), a protein, an aptamer and small molecule, ribosome, RNAi agent, guide RNA (gRNA) and peptide nucleic acid (PNA) and analogues or variants thereof. In one embodiment, the agent is a transcriptional activation system (e.g., a gRNA for use in a gene activation system such as CRISPR/Cas or TALEN) for increasing the expression of the one or more endogenous transcription factors.

The method of inducing differentiation of the cell population (i.e., source cells), may comprise delivering to the cells a nucleic acid comprising an open reading frame encoding one or more of the polypeptides having transcription factor activity and/or one or more of the transcription factors (e.g., in an expression cassette), the transcription factor protein and/or polypeptides themselves, or an activator of transcription of the open reading frame encoding the polypeptide and/or transcription factor. This results in the amount of the transcription factor in the cells being increased, and the cells differentiate to form pancreatic beta cells. Said open reading frame may be part of a recombinant expression cassette.

In one embodiment, the nucleic acid comprises a recombinant or exogenous expression cassette comprising the transcription factor sequences (or genes) in a sufficient number to cause cellular reprogramming of source cells to pancreatic beta cells. The exogenous expression cassette may comprise an externally inducible transcriptional regulatory element for inducible expression of the one or more transcription factors, such as an inducible promoter, e.g., comprising a tetracycline response element or variant thereof.

If expression of the transcription factors is increased by introducing an exogenous sequence encoding the transcription factor (e.g., the transcription factor gene), then it would be understood that any suitable system for delivering the sequence may be used. The gene delivery system may be a transposon system; a viral gene delivery system; an episomal gene delivery system; or a homologous recombination system such as utilizing a zinc finger nuclease, a transcription activator-like effector nuclease (TALENs), a meganuclease, or

CRISPR/Cas, or the like.

Alternatively, introduction of a nucleic acid, such as DNA or RNA, into cells may use any suitable methods for nucleic acid delivery for transformation of a cell, as described herein or as would be known to one of ordinary skill in the art. Such methods include, but are not limited to, direct delivery of DNA such as by ex vivo transfection, by injection (including microinjection), by electroporation, by calcium phosphate precipitation, by using DEAE-dextran followed by polyethylene glycol, by direct sonic loading, by liposome mediated transfection, by receptor-mediated transfection, by microprojectile bombardment, by agitation with silicon carbide fibers, by Agrobacterium-mediated transformation, and any combination of such methods. Through the application of these techniques, cells may be stably or transiently transformed.

Further, the expression cassette (e.g., an inducible recombinant expression cassette) may include cleavable sequences. Such sequences are sequences that are recognised by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included, but preferably two or more are present. These cleavable sequences may be at any suitable point in the cassette, such that a selected portion of the cassette, or the entire cassette, can be selectively removed if desired. The cleavable sites may thus flank the part/all of the genetic sequence that it may be desired to remove. The method may therefore also comprise removal of the expression cassette and/or the genetic material.

In an alternative embodiment, the cell population is contacted with one or more agents that has the same effect as activating or increasing the expression or amount of the transcription factors (i.e. an indirect method of increasing the expression transcription factor). In this aspect of the invention, the method comprises introducing an exogenous agent which mimics the effect of increasing the expression of the transcription factors described herein. For example, such a method may comprise introducing a protein (e.g. an engineered zinc finger nuclease) that has a DNA-binding activity analogous to the transcription factor. For instance, PPAR proteins bind to peroxisome proliferator responsive elements, so the activity of these transcription factors could be reproduced by a zinc finger nuclease engineered to bind the same domain.

It will be understood that a combination of one or more of the methods for expressing the polypeptides having transcription factor activity or increasing the expression of the transcription factors may be used where the combination overall results in activity necessary for the forward programming to pancreatic beta cells.

### Vectors

In one embodiment, the polypeptides having transcription factor activity or the transcription factors themselves (e.g., combinations of polypeptides and/or transcription factors) are introduced into the cell population using a vector. One of skill in the art would be well equipped to construct a vector through standard recombinant techniques. Vectors include but are not limited to plasmids, cosmids, viruses (bacteriophage, animal viruses, and plant viruses), and artificial chromosomes (e.g., YACs).

In one embodiment, a nucleic acid sequence encoding one or more transcription factors is introduced into a cell by a transposon system (i.e. involving a transposon plasmid). A transposon delivery system is comprised of two plasmids, one encoding the transposase and one encoding the transcription factor(s). The transposase protein mediates random integration of the transcripts encoded in the transposon plasmid into the genome. In one embodiment, the transposon system is selected from a PiggyBac or Sleeping Beauty transposon system. The transposon plasmid encodes a payload flanked by two ITRs (internal terminal repeats). The payload may comprise a Tet inducible promoter, the transcription factor(s), and optionally a selection marker, e.g. an antibiotic selection cassette under a constitutive promoter.

In one embodiment, the transposase and transposon plasmids are delivered by nucleofection or lipofection into the cells. The number of integration events, and therefore the number of copies of payload per cell can be in part controlled by adjusting the total and relative amounts of transposase and transposon plasmid DNA. This allows the combinatorial delivery of transcription factors at a single cell level.

In one embodiment, the vector is a viral vector. The viral gene delivery system may be an RNA-based or DNA-based viral vector. Viral vectors include retroviral vectors, lentiviral vectors (e.g., derived from HIV-1, HIV-2, SIV, BIV, FIV etc.), gammaretroviral vectors, adenoviral (Ad) vectors (including replication competent, replication deficient and gutless forms thereof), adeno-associated virus-derived (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumour virus vectors, Rous sarcoma virus vectors and Sendai virus vectors. In a further embodiment, the viral vector is selected from: a lentiviral vector, an adeno-associated virus vector or a Sendai virus vector. In a yet further embodiment, the viral vector is a lentiviral vector.

Lentiviral vectors are well known in the art. Lentiviral vectors are complex retroviruses capable of integrating randomly into the host cell genome, which, in addition to the common retroviral genes gag, pol, and env, contain other genes with regulatory or structural function (e.g., accessory genes Vif, Nef, Vpu, Vpr). Lentiviral vectors have the advantage of being able to infect non-dividing cells and can be used for both in vivo and ex vivo gene transfer and expression of nucleic acid sequences. For example, recombinant lentiviral vector capable of infecting a non-dividing cell wherein a suitable host cell is transfected with two or more vectors carrying the packaging functions, namely gag, pol and env, as well as rev and tat.

In one embodiment, the vector is a self-replicating RNA vector expression system. For example, the system may comprise self-replicating RNA vectors that remain ectopic to the host cell genome and encode the transcription factors that induce reprogramming. Self-replicating RNA vectors are known in the art and many are based on positive strand RNA viruses, such as alphaviruses.

In one embodiment, the viral vector is used at a high multiplicity of infection (MOI). A high MOI helps to ensure that more than one transcription factor is introduced into the source cell. In one embodiment, the MOI is greater than 0.5, such as 1.0 or above.

In one embodiment, a nucleic acid sequence encoding the one or more polypeptides having transcription factor activity and/or transcription factors is introduced into a cell by a plasmid. In one embodiment, at least one nucleic acid sequence encoding the polypeptides having transcription factor activity and/or the transcription factors is introduced into a cell on a single plasmid.

In one embodiment, the plasmid is episomal. Episomal vectors are able to introduce large fragments of DNA into a cell but are maintained extra-chromosomally, replicated once per cell cycle, partitioned to daughter cells efficiently, and elicit substantially no immune response. In alternative embodiments, an Epstein-Barr virus (EBV)-based episomal vector, a yeast-based vector, an adenovirus-based vector, a simian virus 40 (SV40)-based episomal vector, or a bovine papilloma virus (BPV)-based vector may be used.

Site-specific delivery

Any suitable technique for insertion of a nucleic acid sequence into a specific sequence may be used, and several are described in the art. Suitable techniques include any method which introduces a break at the desired location and permits recombination of the vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR).

Several techniques exist to allow customized site-specific generation of DSB in the genome. Many of these involve the use of customized endonucleases, such as zinc finger nucleases, TALENs or the clustered regularly interspaced short palindromic repeats/CRISPR associated protein (CRISPR/Cas, e.g. CRISPR/Cas9) system.

Zinc finger nucleases are artificial enzymes which are generated by fusion of a zinc-finger DNA-binding domain to the nuclease domain of the restriction enzyme Fokl. The latter has a non-specific cleavage domain which must dimerise in order to cleave DNA. This means that two zinc finger nuclease monomers are required to allow dimerisation of the Fokl domains and to cleave the DNA. The DNA binding domain may be designed to target any genomic sequence of interest, is a tandem array of Cys2His2 zinc fingers, each of which recognises three contiguous nucleotides in the target sequence. The two binding sites are separated by 5-7 bp to allow optimal dimerization of the Fokl domains. The enzyme thus is able to cleave DNA at a specific site, and target specificity is increased by ensuring that two proximal DNA-binding events must occur to achieve a double-strand break.

Transcription activator-like effector nucleases, or TALENs, are dimeric transcription factor/nucleases. They are made by fusing a TAL effector DNA-binding domain to a DNA cleavage domain (a nuclease). Transcription activator-like effectors (TALEs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition. This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of modules, almost any sequence may be targeted. Again, the nuclease used may be Fokl or a derivative thereof.

Three types of CRISPR mechanisms have been identified, of which type II is the most studied. The CRISPR/Cas9 system (type II) utilises the Cas9 nuclease to make a double-stranded break in DNA at a site determined by a short guide RNA. The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognize and cut the exogenous genetic elements using RNA interference. The CRISPR immune response occurs through two steps: CRISPR-RNA (crRNA) biogenesis and crRNA-guided interference. CrRNA molecules are composed of a variable sequence transcribed from the protospacer DNA and a CRISPR repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: the Cas9 nuclease itself and a gRNA. The gRNA is a fusion of a customised, site-specific crRNA (directed to the target sequence) and a standardised tracrRNA.

Once a DSB has been made, a donor template with homology to the targeted locus is supplied; the DSB may be repaired by the homology-directed repair (HDR) pathway allowing for precise insertions to be made.

Derivatives of this system are also possible. Mutant forms of Cas9 are available, such as Cas9D10A, with only nickase activity. This means it cleaves only one DNA strand, and does not activate NHEJ. Instead, when provided with a homologous repair template, DNA repairs are conducted via the high-fidelity HDR pathway only. Cas9D10A may be used in paired Cas9 complexes designed to generate adjacent DNA nicks in conjunction with two sgRNAs complementary to the adjacent area on opposite strands of the target site, which may be particularly advantageous.

The elements for making the double-strand DNA break may be introduced in one or more vectors, such as plasmids, for expression in the cell.

Thus, any method of making specific, targeted double strand breaks in the genome in order to effect the insertion of a gene/inducible cassette may be used in the method of the invention. It may be preferred that the method for inserting the gene/inducible cassette utilises any one or more of zinc finger nucleases, TALENs and/or CRISPR/Cas9 systems or any derivative thereof.

Once the DSB has been made by any appropriate means, the gene/inducible cassette for insertion may be supplied in any suitable fashion as described below. The gene/inducible cassette and associated genetic material form the donor DNA for repair of the DNA at the DSB and are inserted using standard cellular repair machinery/pathways. How the break is initiated will alter which pathway is used to repair the damage, as noted above.

Other methods in the art for site specific delivery include the use of homologous recombination (HR) and recombinase mediated cassette exchange (RMCE). DNA damage mediated site specific insertion methods (such as CRISPR/Cas) can also be used to perform site specific integration of DNA recognition sequences (‘att’ sites) which in turn mediate site specific insertion via the activity of tyrosine and serine recombinases or integrases. These sites (e.g. attP) once inserted into the genome, can mediate site specific HR and RMCE. Insertion of exogenous nucleic acid sequences occurs through homologous recombination between cognate attP and attB sites mediated by the expression of the appropriate and cognate recombinase (e.g. Flp, Cre) or integrase (PhiC31, Bxb1). Using targeting vectors, as described above, flanked by attB sites, site specific exogenous DNA insertion of transgenes can be achieved.

### Controlled Expression

In one embodiment, expression of the transcription factors is under inducible control. In this aspect of the invention, the transcription and translation (expression) of the polypeptides having transcription factor activity and/or the transcription factors may be controlled within the cell. This permits overexpression of the transcription factor(s), if required.

An exogenous expression cassette carrying the transcription factors may comprise an externally inducible transcriptional regulatory element (i.e., an inducible promoter) for rapid induction of protein expression in response to external stimuli, i.e. inducible gene (or transgene) expression. The presence or addition of the appropriate external stimuli (e.g. protein, compound or chemical) to cell culture media modulates the controlled expression of the genetic sequence within the inducible expression cassette; and may be administered continuously or transiently to modulate transcription as required.

Expression of the transcription factors described herein may be increased using a dual cassette expression system, such as the system described in WO2018096343, which is incorporated herein by reference. In this instance, induced transgene over-expression is achieved by using the Tet-ON system components with transgene expression controlled by doxycycline. The components are split between two genomic safe harbour sites (GSH) to reduce the risk of epigenetic gene silencing. The components are (i) transcriptional activator protein (reverse tetracycline trans-activator (rtTA)), which in the presence of doxycycline binds (ii) tetracycline response element (TRE; multiple TetO repeat sequences & minimal

Cytomegalovirus (CMV) promoter). TRE binding by rtTA trans-activates transgene expression. Trans-activatable coding sequences for transgenes may be of human origin.

Therefore, in one embodiment, a sequence encoding one or more of the polypeptides having transcription factor activity and/or the transcription factors is introduced into the cell population using a method comprising:
insertion of a coding sequence for a transcriptional regulator protein into a first genomic safe harbour site of a source cell present in the cell population; and
insertion of one or more inducible cassettes into one or more second genomic safe harbour sites of the source cell, wherein said one or more inducible cassettes comprises said sequence encoding the one or more polypeptides having the activity of one or more transcription factors and/or the transcription factors operably linked to an inducible promoter, and said promoter is regulated by the transcriptional regulator protein.

In one embodiment, a sequence encoding one or more (e.g., two or more or three or more) polypeptides having transcription factor activity and/or of the transcription factors is introduced into the cell population using a method comprising:
targeted insertion of a coding sequence for a transcriptional regulator protein into a first genomic safe harbour site of a source cell present in the cell population; and
targeted insertion of an inducible cassette into a second genomic safe harbour site of the source cell, wherein said inducible cassette comprises said sequence encoding the one or more polypeptides having the activity of one or more transcription factors and/or the one or more transcription factors operably linked to an inducible promoter, and said promoter is regulated by the transcriptional regulator protein.

According to another aspect of the invention, there is provided a dual expression system comprising:
(a) a first expression cassette comprising a gene encoding a transcriptional regulator protein flanked by one or more homology arms targeting the first expression cassette to a first genomic safe harbour site; and
(b) a second expression cassette comprising a sequence encoding one or more polypeptides having the activity of one or more transcription factors and/or transcription factors operably linked to an inducible promoter, flanked by one or more homology arms targeting the second expression cassette to a second genomic safe harbour site, wherein the inducible promoter is regulated by the transcriptional regulator protein of the first expression cassette, and wherein the one or more transcription factors are selected from the group consisting of: GLIS3, PDX1, NEUROD1, NKX6-2, ONECUT1 and variants thereof. In one embodiment, the transcription factors comprise PDX1 and GLIS3. In a further embodiment, the transcription factors comprise PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors comprise PDX1, GLIS3 and NEUROD1, preferably in combination with FOXK1 and/or ONECUT1.

This embodiment of the invention provides a dual expression cassette system. The insertion of the gene encoding a transcriptional regulator protein into the first GSH provides the control mechanism for the expression of the inducible cassette which is operably linked to the inducible promoter and inserted into a second GSH site. In one embodiment, the first and second GSH are different (i.e. are located at different positions in the genome). It will be understood that if more than one transcription factor is to be introduced into the cell using the dual expression system, then the transcription factors may be introduced into the second GSH site (i.e. a multicistronic cassette at the same GSH site), or into multiple GSH sites (i.e. as separate cassettes across different GSH sites).

Alternatively, the dual expression cassette system utilises different alleles of the same GSH. In this embodiment, the inducible cassette may be inserted into one allele of the GSH and the system controlling the expression of the inducible cassette into the other allele of the GSH (e.g. as described in DeKelver et al., 2010, Genome Res., 20, 1133-43 and Qian et al., 2014, Stem Cells, 32, 1230-8).

A GSH site is a locus within the genome wherein a gene or other genetic material may be inserted without any deleterious effects on the cell or on the inserted genetic material. Most beneficial is a GSH site in which expression of the inserted gene sequence is not perturbed by any read-through expression from neighbouring genes and expression of the inducible cassette minimizes interference with the endogenous transcription programme. More formal criteria have been proposed that assist in the determination of whether a particular locus is a GSH site in future (Papapetrou et al. (2011) Nature Biotechnology, 29 (1): 73-8). These criteria include a site that is (i) 50 kb or more from the 5′ end of any gene, (ii) 300 kb or more from any gene related to cancer, (iii) 300 kb or more from any microRNA (miRNA), (iv) located outside a transcription unit and (v) located outside ultraconserved regions (UCR). It may not be necessary to satisfy all of these proposed criteria, since GSH already identified do not fulfil all of the criteria. It is thought that a suitable GSH will satisfy at least 2, 3, 4 or all of these criteria. Any suitable GSH site may be used in the method of the invention, on the basis that the site allows insertion of genetic material without deleterious effects to the cell and permits transcription of the inserted genetic material. Those skilled in the art may use these simplified criteria to identify a suitable GSH, and/or the more formal criteria set out above.

Insertion of the coding sequence for a transcriptional regulator protein and/or the inducible cassette may be carried out through direct delivery methods as described above. It is understood that although such direct delivery methods may lead to the random insertion of the genetic material, screening may be carried out in order to identify clones that show no deleterious effects, are able to express the genetic material and are able to be forward programmed or reprogrammed to pancreatic beta cells, and by doing so one is able to confirm that the transcriptional regulator protein/inducible cassette has been inserted into a GSH site.

In one embodiment the insertion of the transcriptional regulator protein or the inducible cassette is targeted. In a further embodiment, the insertion of the transcriptional regulator protein and the inducible cassette is targeted. “Targeted insertion”, as with site-specific delivery, is understood as the insertion of the genetic material into a pre-chosen GSH. As discussed above, this can be carried out using techniques known in the art such as zinc finger nucleases, TALENs or the clustered regularly interspaced short palindromic repeats/CRISPR associated protein (CRISPR/Cas, e.g. CRISPR/Cas9) system.

In one embodiment, the first and second genomic safe harbour sites (GSHs) are selected from (in particular any two) of the ROSA26 locus, the AAVS1 locus, the CLYBL gene, the CCR5 gene or the HPRT gene. Insertions specifically within genomic safe harbour sites is preferred over random genome integration, since this is expected to be a safer modification of the genome, and is less likely to lead to unwanted side effects such as silencing natural gene expression or random insertional mutagenesis.

The adeno-associated virus integration site 1 locus (AAVS1) is located within the protein phosphatase 1, regulatory subunit 12C (PPP1R12C) gene on human chromosome 19, which is expressed uniformly and ubiquitously in human tissues. AAVS1 has been shown to be a favourable environment for transcription, since it comprises an open chromatin structure and native chromosomal insulators that enable resistance of the inducible cassettes against silencing. There are no known adverse effects on the cell resulting from disruption of the PPP1R12C gene. Moreover, an inducible cassette inserted into this site remains transcriptionally active in many diverse cell types.

The human ROSA26 (hROSA26) site has been identified on the basis of sequence analogy with a GSH from mice (ROSA26—reverse oriented splice acceptor site #26). The hROSA26 locus is on chromosome 3 (3p25.3), and can be found within the Ensembl database (GenBank:CR624523). The integration site lies within the open reading frame (ORF) of the THUMPD3 long non-coding RNA (reverse strand). Since the hROSA26 site has an endogenous promoter, the inserted genetic material may take advantage of that endogenous promoter, or alternatively may be inserted operably linked to a promoter.

Intron 2 of the Citrate Lyase Beta-like (CLYBL) gene, on the long arm of Chromosome 13, was identified as a suitable GSH since it is one of the identified integration hot-spots of the phage derived phiC31 integrase. Studies have demonstrated that randomly inserted inducible cassettes into this locus are stable and expressed. It has been shown that insertion of inducible cassettes at this GSH do not perturb local gene expression (Cerbini et al. (2015) PLOS One, 10(1): e0116032). CLYBL thus provides a GSH which may be suitable for use in the present invention. CCR5, which is located on chromosome 3 (position 3p21.31) is a gene which codes for HIV-1 major co-receptor. Interest in the use of this site as a GSH arises from the null mutation in this gene that appears to have no adverse effects, but predisposes to HIV-1 infection resistance. Zinc-finger nucleases that target the third exon have been developed, thus allowing for insertion of genetic material at this locus.

The hypoxanthine-guanine phosphoribosyltransferase (HPRT) gene encodes a transferase enzyme that plays a central role in the generation of purine nucleotides through the purine salvage pathway.

Other GSH have been described in the art, such as in Sadelain et al. (2012) Nature Reviews 12:51-58 and in WO2021/152086, which are herein incorporated by reference.

GSH in other organisms have been identified and include ROSA26, HRPT and Hipp11 (H11) loci in mice. Mammalian genomes may include GSH sites based upon pseudo attP sites. For such sites, hiC31 integrase, the Streptomyces phage-derived recombinase, has been developed as a non-viral insertion tool, because it has the ability to integrate an inducible cassette-containing plasmid carrying an attB site into pseudo attP sites.

Technically, the insertions into the first and/or second GSH may occur on one chromosome, or on both chromosomes. The GSH exists at the same genetic loci on both chromosomes of diploid organisms. Insertion within both chromosomes is advantageous since it may enable an increase in the level of transcription from the inserted genetic material within the inducible cassette, thus achieving particularly high levels of transcription.

Specific insertion of genetic material into the particular GSH based upon customised site-specific generation of DNA double-strand breaks at the GSH may be achieved. The genetic material may then be introduced using any suitable mechanism, such as homologous recombination. Any method of making a specific DSB in the genome may be used, but preferred systems include CRISPR/Cas9 and modified versions thereof, zinc finger nucleases and the TALEN system, or via HR or RCME mediated integration or recombination.

One or more genetic sequences may be controllably transcribed from within the second and/or further GSH. Indeed, the inducible cassette may contain 1, 2, 3, 4, 5, 6, 7, 8, 9 or 10 genetic sequences (e.g., transcription factor sequences) which it is desired to insert into the GSH and the transcription of which be controllably induced. Therefore, the transcription factors required by the present invention may be included within the same cassette introduced into the second genomic safe harbour site. For example, three or more transcription factors may be included in, for example, three mono-cistronic constructs, one mono-cistronic and one bi-cistronic construct or one tri-cistronic construct. It will be understood that similar combinations of constructs may be used to achieve higher orders of transcription factor expression.

Alternatively, if a combination of transcription factors is used, the individual transcription factors may be introduced into separate GSHs and/or under the control of the same, different or orthogonal inducible promoters. Therefore, in one embodiment, the transcription factors are introduced into separate GSHs. For example, this may be achieved by utilising three or more different GSH sites for three or more transcription factors (i.e., wherein the transcription factors are introduced as mono-cistronic cassettes). Alternatively, this may be achieved by utilising the fact that a GSH exists at the same genetic loci on both chromosomes of diploid organisms, e.g., introducing one transcription factor into the GSH on one chromosome and a different transcription factor into the same GSH on the other chromosome. This embodiment is advantageous if different expression levels or timing of expression of the transcription factors is desired. In one embodiment, the method comprises targeted insertion of the transcription factors, each operably linked to an inducible promoter into a second, third and fourth genomic safe harbour site of the source cell. The inducible promoter may be the same of each transcription factor and therefore are all regulated by the transcriptional regulator protein.

A transcriptional regulator protein is a protein that binds to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor).

The DNA sequence that a transcriptional regulator protein binds to is called a transcription factor-binding site or response element, and these are found in or near the promoter of the regulated DNA sequence. Transcriptional activator proteins bind to the response element and promote gene expression. Such proteins are preferred in the methods of the present invention for controlling inducible cassette expression. Transcriptional repressor proteins bind to the response element and prevent gene expression.

Transcriptional regulator proteins may be activated or deactivated by a number of mechanisms including binding of a substance, interaction with other transcription factors (e.g., homo- or hetero-dimerization) or coregulatory proteins, phosphorylation, and/or methylation. The transcriptional regulator protein may be controlled by activation or deactivation.

If the transcriptional regulator protein is a transcriptional activator protein, it is preferred that the transcriptional activator protein requires activation. This activation may be through any suitable means, but it is preferred that the transcriptional regulator protein is activated through the addition to the cell of an exogenous substance. The supply of an exogenous substance to the cell can be controlled, and thus the activation of the transcriptional regulator protein can be controlled. Alternatively, an exogenous substance can be supplied in order to deactivate a transcriptional regulator protein, and then supply withdrawn in order to activate the transcriptional regulator protein.

If the transcriptional regulator protein is a transcriptional repressor protein, it is preferred that the transcriptional repressor protein requires deactivation. Thus, a substance is supplied to prevent the transcriptional repressor protein repressing transcription, and thus transcription is permitted.

Any suitable transcriptional regulator protein may be used, preferably one that may be activated or deactivated. It is preferred that an exogenous substance may be supplied to control the transcriptional regulator protein. Such transcriptional regulator proteins are also called inducible transcriptional regulator proteins.

Tetracycline-Controlled Transcriptional Activation is a method of inducible gene expression where transcription is reversibly turned on or off in the presence of the antibiotic tetracycline or one of its derivatives (e.g., doxycycline which is more stable). In this system, the transcriptional activator protein is reverse tetracycline-controlled transactivator (rtTa, which may also be referred to as tetracycline-responsive transcriptional activator protein) or a derivative thereof. The rtTA protein is able to bind to DNA at specific TetO operator sequences. Several repeats of such TetO sequences are placed upstream of a minimal promoter (such as the CMV promoter), which together form a tetracycline response element (TRE). There are two forms of this system, depending on whether the addition of tetracycline or a derivative activates (Tet-On) or deactivates (Tet-Off) the rtTA protein.

In a Tet-Off system, tetracycline or a derivative thereof binds rtTA and deactivates the rtTA, rendering it incapable of binding to TRE sequences, thereby preventing transcription of TRE-controlled genes. This system was first described in Gossen et al. (1992) PNAS 89 (12): 5547-5551.

The Tet-On system is composed of two components; (1) the constitutively expressed reverse tetracycline-controlled transactivator (rtTa) and the rtTa-sensitive inducible promoter (Tet Responsive Element, TRE). This may be bound by tetracycline or its more stable derivatives, including doxycycline (dox), resulting in activation of rtTa, allowing it to bind to TRE sequences and inducing expression of TRE-controlled genes. The use of this may be preferred in the method of the invention.

Thus, the transcriptional regulator protein may thus be a reverse tetracycline-controlled transactivator (rtTa) protein, which can be activated or deactivated by the antibiotic tetracycline or one of its derivatives, which are supplied exogenously. If the transcriptional regulator protein is rtTA, then the inducible promoter inserted into the second GSH site includes the tetracycline response element (TRE). The exogenously supplied substance is the antibiotic tetracycline or one of its derivatives.

Variants and modified rtTa proteins may also be used in the methods of the invention, these include Tet-On Advanced transactivator (also known as rtTA2S-M2) and Tet-On 3G (also known as rtTA-V16, derived from rtTA2S-S2).

The tetracycline response element (TRE) generally consists of 7 repeats of the 19 bp bacterial TetO sequence separated by spacer sequences, together with a minimal promoter. Variants and modifications of the TRE sequence are possible, since the minimal promoter can be any suitable promoter. Preferably the minimal promoter shows no or minimal expression levels in the absence of rtTa binding. The inducible promoter inserted into the second GSH may thus comprise a TRE.

A modified system based upon tetracycline control is the T-REX System (Thermo-Fisher Scientific), in which the transcriptional regulator protein is a transcriptional repressor protein, TetR. The components of this system include (i) an inducible promoter comprising a strong human cytomegalovirus immediate-early (CMV) promoter and two tetracycline operator 2 (TetO2) sites, and a Tet repressor (TetR). In the absence of tetracycline, the Tet repressor forms a homodimer that binds with extremely high affinity to each TetO2 sequence in the inducible promoter, and prevent transcription from the promoter. Once added, tetracycline binds with high affinity to each Tet repressor homodimer rendering it unable to bind to the Tet operator. The Tet repressor: tetracycline complex then dissociates from the Tet operator and allows induction of expression. In this instance, the transcriptional regulator protein is TetR and the inducible promoter comprises two TetO2 sites. The exogenously supplied substance is tetracycline or a derivative thereof.

Other inducible expression systems are known and can be used in the method of the invention. These include the Complete Control Inducible system from Agilent Technologies. This is based upon the insect hormone ecdysone or its analogue ponasterone A (ponA) which can activate transcription in mammalian cells which are transfected with both the gene for the Drosophila melanogaster ecdysone receptor (EcR) and an inducible promoter comprising a binding site for the ecdysone receptor. The EcR is a member of the retinoid-X-receptor (RXR) family of nuclear receptors. In humans, EcR forms a heterodimer with RXR that binds to the ecdysone-responsive element (EcRE). In the absence of PonA, transcription is repressed by the heterodimer.

Thus, the transcriptional regulator protein can be a repressor protein, such as an ecdysone receptor or a derivative thereof. Examples of the latter include the VgEcR synthetic receptor from Agilent technologies which is a fusion of EcR, the DNA binding domain of the glucocorticoid receptor and the transcriptional activation domain of Herpes Simplex Virus VP16. The inducible promoter comprises the EcRE sequence or modified versions thereof together with a minimal promoter. Modified versions include the E/GRE recognition sequence of Agilent Technologies, in which mutations to the sequence have been made. The E/GRE recognition sequence comprises inverted half-site recognition elements for the retinoid-X-receptor (RXR) and GR binding domains. In all permutations, the exogenously supplied substance is ponasterone A, which removes the repressive effect of EcR or derivatives thereof on the inducible promoter, and allows transcription to take place.

Alternatively, inducible systems may be based on the synthetic steroid mifepristone as the exogenously supplied substance. In this scenario, a hybrid transcriptional regulator protein is inserted, which is based upon a DNA binding domain from the yeast GAL4 protein, a truncated ligand binding domain (LBD) from the human progesterone receptor and an activation domain (AD) from the human NF-κB. This hybrid transcriptional regulator protein is available from Thermo-Fisher Scientific (Gene Switch™). Mifepristone activates the hybrid protein, and permits transcription from the inducible promoter which comprises GAL4 upstream activating sequences (UAS) and the adenovirus E1b TATA box. This system is described in Wang et al. (1994) PNAS 91:8180-8184.

The transcriptional regulator protein can thus be any suitable regulator protein, either an activator or repressor protein. Suitable transcriptional activator proteins are tetracycline-responsive transcriptional activator protein or the Gene Switch hybrid transcriptional regulator protein. Suitable repressor proteins include the Tet-Off version of rtTA, TetR or EcR. The transcriptional regulator proteins may be modified or derivatised as required.

The inducible promoter can comprise elements which are suitable for binding or interacting with the transcriptional regulator protein. The interaction of the transcriptional regulator protein with the inducible promoter is preferably controlled by the exogenously supplied substance.

The exogenously supplied substance can be any suitable substance that binds to or interacts with the transcriptional regulator protein. Suitable substances include tetracycline (or derivatives thereof, such as doxycycline), ponasterone A and mifepristone.

It is preferred that the gene encoding the transcriptional regulator protein is operably linked to a constitutive promoter. Alternatively, the first GSH can be selected such that it already has a constitutive promoter than can also drive expression of the transcriptional regulator protein gene and any associated genetic material. Constitutive promoters ensure sustained and high-level gene expression. Commonly used constitutive promoters, including the human β-actin promoter (ACTB), cytomegalovirus (CMV), elongation factor-1α, (EF1α), phosphoglycerate kinase (PGK) and ubiquitin C (UbC). The CAG promoter is a strong synthetic promoter frequently used to drive high levels of gene expression and was constructed from the following sequences: (C) the cytomegalovirus (CMV) early enhancer element, (A) the promoter, the first exon and the first intron of chicken beta-actin gene, and (G) the splice acceptor of the rabbit beta-globin gene.

According to a further aspect of the invention, there is provided a method for the production of pancreatic beta cells from a source cell, comprising the steps of:
a) insertion of a gene encoding a transcriptional regulator protein into a first genomic safe harbour site of the source cell; and
b) insertion of at least one nucleotide sequence encoding one or more polypeptides having the activity of one or more transcription factors and/or one or more transcription factors, the transcription factors selected from the group consisting of: GLIS3, PDX1, NEUROD1, NKX6-2, ONECUT1 and variants thereof, operably linked to an inducible promoter into a second genomic safe harbour site of the source cell, wherein said inducible promoter is regulated by the transcriptional regulator protein; and
c) culturing the source cell(s) comprising the insertions to obtain pancreatic beta cells.

According to a further aspect of the invention, there is provided a method for the production of pancreatic beta cells from a source cell, comprising the steps of:
a) targeted insertion of a gene encoding a transcriptional regulator protein into a first genomic safe harbour site of the source cell; and
b) targeted insertion of at least one nucleotide sequence encoding one or more polypeptides having the activity of one or more transcription factors and/or one or more transcription factors, the transcription factors selected from the group consisting of: GLIS3, PDX1, NEUROD1, NKX6-2, ONECUT1 and variants thereof, operably linked to an inducible promoter into a second genomic safe harbour site of the source cell, wherein said inducible promoter is regulated by the transcriptional regulator protein; and
c) culturing the source cell(s) comprising the insertions to obtain pancreatic beta cells.

According to a further aspect of the invention, there is provided a method for the production of pancreatic beta cells from a source cell, comprising the steps of:
a) insertion of a gene encoding a transcriptional regulator protein into a first genomic safe harbour site of the source cell; and
b) insertion of at least one nucleotide sequence encoding one of more polypeptides having the activity of one or more transcription factors and/or one or more transcription factors, the transcription factors selected from the group consisting of: GLIS3, PDX1, NEUROD1, NKX6-2, ONECUT1 and variants thereof, operably linked to an inducible promoter into one or more further genomic safe harbour sites of the source cell, wherein said inducible promoter is regulated by the transcriptional regulator protein; and
c) culturing the source cell(s) comprising the insertions to obtain pancreatic beta cells.

According to a further aspect of the invention, there is provided a method for the production of pancreatic beta cells from a source cell, comprising the steps of:
a) targeted insertion of a gene encoding a transcriptional regulator protein into a first genomic safe harbour site of the source cell; and
b) targeted insertion of at least one nucleotide sequence encoding one of more polypeptides having the activity of one or more transcription factors and/or one or more transcription factors, the transcription factors selected from the group consisting of: GLIS3, PDX1, NEUROD1, NKX6-2, ONECUT1 and variants thereof, operably linked to an inducible promoter into one or more further genomic safe harbour sites of the source cell, wherein said inducible promoter is regulated by the transcriptional regulator protein; and
c) culturing the source cell(s) comprising the insertions to obtain pancreatic beta cells.

It will be understood that these aspects of the invention may be used with any of the combinations of transcription factors described herein. In particular, in one embodiment, the transcription factors comprise PDX1 and GLIS3. In a further embodiment, the transcription factors comprise PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors comprise PDX1, GLIS3 and NEUROD1, preferably in combination with FOXK1 and/or ONECUT1.

### Obtaining Pancreatic Beta Cells

In one embodiment, the method additionally comprises monitoring the cell population for at least one characteristic of a pancreatic beta cell. Cells may be monitored throughout culturing to identify expression of key lineage markers.

For example, monitoring may be through the use of engineered ‘reporter’ cell lines (i.e. endogenously tagged proteins or positive selection markers under the control of pancreatic beta cell-specific promoters) or immunostaining and detection, using fluorescence microscopy or flow cytometry. Such material includes genes for markers or reporter molecules, such as genes that induce visually identifiable characteristics including fluorescent and luminescent proteins. Examples include the gene that encodes jellyfish green fluorescent protein (GFP), which causes cells that express it to glow green under blue/UV light, luciferase, which catalyses a reaction with luciferin to produce light, and the red fluorescent protein from the gene dsRed.

The cell may further comprise a positive selection marker and/or selectable reporter expression cassette, e.g., comprising a pancreatic beta cell-specific promoter operably linked to a reporter gene.

Selectable markers may include resistance genes to antibiotics or other drugs. Examples of drug resistance genes may include: a puromycin resistance gene, an ampicillin resistance gene, a neomycin resistance gene, a tetracycline resistance gene, a kanamycin resistance gene or a chloramphenicol resistance gene. Cells can be cultured on a medium containing the appropriate drug (i.e., a selection medium) and only those cells which incorporate and express the drug resistance gene will survive. Therefore, by culturing cells using a selection medium, it is possible to select for cells comprising and expressing a drug resistance gene, positively enriching for a target cell population.

Examples of fluorescent protein genes which may be used as markers include: a green fluorescent protein (GFP) gene, yellow fluorescent protein (YFP) gene, red fluorescent protein (RFP) gene or aequorin gene. Cells expressing the fluorescent protein can be detected using a fluorescence microscope and fluorescence activated cell sorting (FACS) used to identify and select cell populations based on the expression of fluorescent proteins.

Fluorescent protein genes may be tagged with a nuclear localization signal peptide to confine expression of the fluorescent proteins to the nucleus. This may be helpful in cell types with a high lipid content which may not be suitable for FACS. This allows end-point fluorescence-activated cell sorting to be carried out on either whole cell populations, or purified nuclei which maintain an intact fluorescent signal.

Examples of chromogenic enzyme genes which may be used as markers, and known in the art, include but are not limited to: β-galactosidase gene, β-glucuronidase gene, alkaline phosphatase gene, or secreted alkaline phosphatase SEAP gene. Cells expressing these chromogenic enzyme genes can be detected by applying the appropriate chromogenic substrate (e.g., X-gal for β galactosidase) so that cells expressing the marker gene will produce a detectable colour (e.g., blue in a blue-white screen test).

The method may therefore comprise a selection or enrichment step for pancreatic beta cells provided from the methods described herein. In one embodiment, the method comprises the step of sorting the pancreatic beta cells using fluorescence activated cell sorting (FACS) or immunomagnetic sorting methods based on the expression of pancreatic beta cell markers and/or absence of non-pancreatic beta cell markers. A labelled binding agent directed to target cell surface proteins may be used. Any binding agent capable of specific binding to a particular epitope may be used for this purpose, for example an antibody or a fragment thereof, a peptide or a synthetic binder such as a plastic antibody, or an aptamer or oligonucleotide, capable of specific binding to an epitope. The binding agent may be labelled with a detectable marker, such as a luminescent, fluorescent (e.g. fluorochrome), enzyme or radioactive marker; alternatively or additionally an affinity tag, e.g. a biotin, avidin, streptavidin or His (e.g. hexa-His) tag. In one embodiment, fluorochrome conjugated antibodies targeting cell surface proteins (e.g. pancreatic beta cell markers) may be used to sort target cells.

In another embodiment, pancreatic beta cells are enriched by drug-resistance selection from genetically engineered source cells expressing an antibiotic-resistance gene under the control of a pancreatic beta cell-specific promoter.

The method may generate cells (i.e., differentiated cells) exhibiting at least one characteristic of a pancreatic beta cell. One or more characteristics may be used to select for the pancreatic beta cells generated by methods of the invention.

Characteristics include but are not limited to the detection or quantitation of expressed cell markers, enzymatic activity, and the characterization of morphological features and intercellular signalling. The biological function of a pancreatic beta cell may also be evaluated, for example using functional assays, e.g. secretion of insulin or amylin.

In one embodiment, the characteristic (i.e., of a pancreatic beta cell, in particular a human pancreatic beta cell) is selected from one or more of:
(i) expression of one or more cell markers, such as proinsulin, MAFA or a combination thereof;
(ii) secretion of insulin or amylin; or
(iii) pancreatic beta cell morphological features.

In one embodiment, the cells are sorted on the basis of acquisition of expression of a mature pancreatic beta cell marker, such as insulin c-peptide.

The pancreatic beta cell markers may be markers obtained by transcriptome analysis. For example, single cell RNA sequencing has been used to provide detailed transcriptional profiles of human pancreatic beta cells obtained from primary human tissues. This information can be used to identify pancreatic beta cells generated by the methods described herein. Additional resources, such as Human Cell Atlas and CellTypist may also be used to identify markers of pancreatic beta cells.

The method may comprise assaying the differentiated cells obtained by the method described herein and determining a set of transcribed genes; comparing the set of transcribed genes of the differentiated cells to one or more reference sets of transcribed genes from one or more reference pancreatic beta cells; and identifying a match between the differentiated cells and a reference pancreatic beta cell.

In one embodiment, the method comprises the step of identifying differentiated cells as a type of pancreatic beta cell by assaying morphological features of the differentiated cells and matching the morphological features to a reference tissue or cell's morphological features.

In one embodiment, the method comprises the step of identifying differentiated cells as a type of pancreatic beta cell by assaying protein marker expression of the differentiated cells and matching the protein marker expression to a reference pancreatic beta cell protein marker expression.

In one embodiment, the method comprises the step of identifying differentiated cells as a type of pancreatic beta cell by assaying a function and matching the function to a function of a reference pancreatic beta cell.

In one embodiment, the cells obtained by the methods of the invention express a pancreatic beta cell phenotype. The target cells may also be negative for markers of pluripotency.

Alternatively, certain differentiated cells may be sorted from other differentiated cells and from cells on the basis of their expression of a lineage-specific cell surface antigen. Yet another means is by assessing expression at the RNA level, e.g., by RT-qPCR methods or by single cell RNA sequencing without any sorting or pre-selection step. Such techniques are known in the art.

### Cell Culturing

In one embodiment, the method includes culturing the cell population for a sufficient time and under conditions to allow differentiation to a pancreatic beta cell. Generally, cells of the present invention are cultured in a culture medium, which is a nutrient-rich buffered solution capable of sustaining cell growth.

The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. For example, the media may comprise Basal Medium (e.g. DMEM/F12 or STEMPRO-34) supplemented with GLUTAMAX, antibiotics (such as penicillin or streptomycin), B27 supplement and/or N2 supplement (all available from Thermo Fisher Scientific). Any suitable growth medium containing sufficient quantities of vitamins, minerals, salts, glucose, amino acids and carrier proteins desirable for cells differentiation may be used, in particular MCDB-131 medium (available from GIBCO). The media may then be further supplemented at different time points during the culturing process. For example, one or more peptide hormones and/or cytokines can be added at 2, 4 and/or 10 days during the culturing process.

In one embodiment, the culture media comprises one or more components selected from the group consisting of: heparin, bovine serum albumin (BSA) preferably without fatty acids, fibroblast growth factor (FGF), bone morphogenetic protein (BMP), retinoic acid, epidermal growth factor (EGF), transforming growth factor-β (TGF-β), insulin-like growth factors 1 and 2, hepatocyte growth factor (HGF), glucagon-like peptide-1 (GLP-1), exendin-4 (a peptide analog of GLP-1), nicotinamide and thyroid hormone.

Pancreatic beta cells may be obtained using methods of the invention at least about 2, 3, 4, 5, 6, 7, 8, 9, 10, 11, 12, 13, 14, 15, 16, 17, 18, 19, or 20 days after culturing. In one embodiment, the method comprises culturing under suitable conditions for at least 3 days, such as at least 7 days or about 10 days. In further embodiments, method comprises culturing cells for a duration (e.g., at least 4 days, at least 5 days, at least 6 days, at least 7 days, at least 8 days, at least 9 days, at least 10 days, at least 11 days, at least 12 days, at least 13 days, at least 14 days, at least 21 days, at least 28 days, or longer, e.g., from 5 days to 40 days, from 7 days to 35 days, from 14 days to 28 days, or about 21 days) which is sufficient to generate pancreatic beta cells. In some embodiments, the cells are cultured for a period of several hours (e.g., about 2, 3, 4, 5, 6, 7, 8, 9, 10, 15, 18, or 21 hours) to about 35 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days). In one embodiment, the method comprises culturing the cells for at least about 5, 10, 15 or 20 days to produce pancreatic beta cells. In one embodiment, the cells are cultured for a period of between 4 and 25 days, such as between 7 and 14 days.

After culturing, the cell population may comprise two cell types. For example, such a cell population may have two cell types including the stem cells and pancreatic beta cells. In one embodiment, the cell population comprises up to 1, 5, 10, 15, 20, 25, 30, 35, 40, 45, 50, 60, 70, 80, 85, 90, 91, 92, 93, 94, 95, 96, 97, 98, 99 or 99.5% (or any intermediate ranges) of pancreatic beta cells in the resulting cell population.

Culturing the cells may either help to induce cells to commit to a more mature phenotype, preferentially promote survival of the mature cells, or have a combination of both these effects.

According to a further aspect of the invention, there is provided a cell obtainable by any one of the methods defined herein.

According to a further aspect of the invention, there is provided a cell comprising one or more exogenous expression cassettes comprising nucleotide sequences encoding at least one or more polypeptides having the activity of one or more transcription factors and/or transcription factors, wherein the one or more transcription factors are selected from the group consisting of: GLIS3, PDX1, NEUROD1, NKX6-2, ONECUT1, and variants thereof. In one embodiment, the transcription factors comprise PDX1 and GLIS3. In a further embodiment, the transcription factors comprise PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors comprise PDX1, GLIS3 and NEUROD1, preferably in combination with FOXK1 and/or ONECUT1.

As described herein, the exogenous expression cassettes encoding the one, two or three or more polypeptides having transcription factor activity and/or transcription factors may be integrated into the genome of the cell. In a further embodiment, exogenous expression cassettes encoding the two or more polypeptides having transcription factor activity and/or transcription factors are integrated into a (specific) target site in the genome of the cell. Alternatively, exogenous expression cassettes encoding the two or more polypeptides having transcription factor activity and/or transcription factors are integrated into a non-specific target site in the genome of the cell.

### Cell Compositions

According to a further aspect, there is provided a pharmaceutical composition comprising the pancreatic beta cells produced by the method as described herein and a pharmaceutically acceptable carrier.

Pharmaceutical compositions may include pancreatic beta cells as described herein in combination with one or more pharmaceutically or physiologically acceptable carrier, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. Cryopreservation solutions which may be used in the pharmaceutical compositions of the invention include, for example, DMSO.

For purposes of manufacture, distribution, and use, the pancreatic beta cells described herein may be supplied in the form of a cell culture or suspension in an isotonic excipient or culture medium, optionally frozen to facilitate transportation or storage.

### Uses of Pancreatic Beta Cells

The cells produced according to any of the methods of the invention have applications in basic and medical research, diagnostic and therapeutic methods. The cells may be used in vitro to study cellular development, provide test systems for new drugs, enable screening methods to be developed, scrutinise therapeutic regimens, provide diagnostic tests and the like. These uses form part of the present invention. Alternatively, the cells may be transplanted into a human or animal patient for diagnostic or therapeutic purposes. The use of the cells in therapy is also included in the present invention.

According to one aspect of the invention, there is provided a pancreatic beta cell as defined herein, for use in in vitro diagnostics or drug screening.

Pancreatic beta cells generated by methods of the invention may find particular use in drug screening. Therefore, in one embodiment, the method additionally comprises contacting the pancreatic beta cells with a test substance and observing a change (e.g., an effect) in the pancreatic beta cells induced by the test substance. The change or effect may be observed using methods known in the art, for example using pharmacological or toxicological assays. In one aspect, the cells may be used in a method of assessing a test substance (e.g., a drug, such as a compound), comprising assaying a pharmacological or toxicological property of the test substance on the pancreatic beta cells provided by the methods described herein. The method may comprise: a) contacting the pancreatic beta cells described herein with the test substance; and b) assaying an effect of the test substance on the pancreatic beta cells.

Assessment of the activity of a candidate molecule may involve combining the pancreatic beta cells described herein with the candidate molecule, determining any change in the morphology, phenotype, or metabolic activity of the pancreatic beta cells that is attributable to the molecule (i.e., compared with a control, such as untreated cells or cells treated with an inert compound), and then correlating the effect of the molecule with the observed change. The screening may be done either because the candidate molecule is designed to have a pharmacological effect on pancreatic beta cells, or because the molecule is designed to have effects elsewhere but there is a need to determine if it has any unintended side effects.

Cytotoxicity can be determined in the first instance by the effect on cell viability, survival, morphology, and leakage of enzymes into the culture medium. More detailed analysis may be conducted to determine whether a test substance affects cell function without causing toxicity.

Alternatively, the cells can be used to assess changes in gene expression patterns caused by a potential drug candidate. In this embodiment, the changes in gene expression pattern from addition of the candidate drug can be compared with the gene expression pattern caused by a control drug with a known effect on pancreatic beta cells.

Therefore, according to a further aspect, there is provided a method for drug screening (e.g., evaluating drug reactivity), comprising a step of using the pancreatic beta cells produced by the method as described herein. According to a further aspect of the invention, there is provided a method of drug screening comprising contacting a pancreatic beta cell generated using the method as defined herein, or a pancreatic beta cell as defined herein, with the drug and observing a change in the pancreatic beta cell induced by the drug.

According to a further aspect of the invention, there is provided the pancreatic beta cell as defined herein for use in therapy.

In one embodiment, the method additionally comprises transplanting the pancreatic beta cells into a patient. In this aspect of the invention, the cells used to generate the pancreatic beta cells may be autologous (i.e., mature cells removed, modified and returned to the same individual) or from a donor (i.e., allogeneic, including a stem cell line). Direct reprogramming of cells into pancreatic beta cells is amenable to the production of autologous and allogeneic pancreatic beta cells.

Therefore, according to a further aspect of the invention there is provided a method of treating a subject having or at risk of a disease or disorder comprising administering to the subject a therapeutically effective amount of pancreatic beta cells generated using the method as defined herein, or pancreatic beta cells as defined herein.

Pancreatic beta cells of the invention may find use in the development for therapies and the treatment of diabetes (e.g., type 1 diabetes or type 2 diabetes), as well as the secondary disorders of pancreas dysfunction (e.g., obesity, hyperlipidemia, hypertension or cardiovascular disease).

In a different aspect, the cells may be used in tissue engineering. Tissue engineering requires the generation of tissue which could be used to replace tissues or even whole organs of a human or animal. Methods of tissue engineering are known to those skilled in the art, but include the use of a scaffold (an extracellular matrix) upon which the cells are applied in order to generate tissues/organs. These methods can be used to generate an “artificial” tissue or organ. Methods of generating tissues may include additive manufacturing, otherwise known as three-dimensional (3D) printing, which can involve directly printing cells to make tissues. The present invention thus provides a method for generating tissues using the cells produced as described in any aspect of the invention.

### Reprogramming Kits

According to a further aspect, there is provided a kit for differentiating a cell into a pancreatic beta cell comprising:
(i) a source cell and an agent that activates or increases the expression or amount of one or more transcription factors; and/or
(ii) one or more expression cassette(s) comprising nucleotide sequences encoding one or more polypeptides having transcription factor activity and/or one or more transcription factors,
wherein the one or more transcription factors are selected from the group consisting of: GLIS3, PDX1, NEUROD1, NKX6-2, ONECUT1 and variants thereof. In one embodiment, the transcription factors comprise PDX1 and GLIS3. In a further embodiment, the transcription factors comprise PDX1, GLIS3 and one or more additional transcription factors selected from the list consisting of: (1) NEUROD1, (2) ONECUT1, (3) MNX1, (4) FOXK1, (5) ASCL1, (6) NEUROD1 in combination with FOXK1 and (7) NEUROD1 in combination with ONECUT1. In a further embodiment, the transcription factors comprise PDX1, GLIS3 and NEUROD1, preferably in combination with FOXK1 and/or ONECUT1.

In one embodiment, the expression cassette comprises an inducible expression construct comprising a sequence encoding one or more polypeptides having transcription factor activity and/or the transcription factors themselves, in particular two or more transcription factors.

As described herein, combinations of polypeptides having transcription factor activity and/or the transcription factors described herein are of particular use in the present invention. If a combination of polypeptides and/or transcription factors is required, these may be encoded on the same or on different expression cassettes. Therefore, in one embodiment, the kit comprises an expression cassette (preferably an inducible expression cassette) encoding one or more polypeptides and/or transcription factors, such as two, three, four or five transcription factors. Preferably, the kit comprises an expression cassette encoding three or more, more preferably four or more, polypeptides and/or transcription factors.

According to a further aspect, there is provided a use of a kit as defined herein, for differentiating a cell into a pancreatic beta cell.

The kit may include one or more articles and/or reagents for performance of the method. For example, one or more transcription factor genes, derivatives, variants or fragments thereof, for use in the methods described herein may be provided in isolated form and may be part of a kit, e.g., in a suitable container such as a vial in which the contents are protected from the external environment.

In one embodiment, the kit additionally comprises at least one source cell, such as a pluripotent stem cell (such as an induced pluripotent stem cell) or a non-pluripotent, non-pancreatic beta cell.

In one embodiment, the kit additionally comprises a medium for culturing the cell and instructions for preparing the enhanced potency cells or reprogrammed pluripotent cells in accordance with the method defined herein.

It will be understood that all embodiments described herein may be applied to all aspects of the invention.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The invention will now be described using the following, non-limiting examples:

### EXAMPLES

### Example 1—Prioritisation Screen

### Methods:

### Plasmid Library Encoding the TFs, Reporter Line and Transposase-Mediated Delivery

A large-scale library of transcription factor (TF) candidates was shortlisted from selected multi-omics datasets by scientific curation based on a tiered transcriptomics analysis of pancreatic beta-labelled cells and applying a suite of computational tools.

Each TF and a small set of negative controls were cloned separately into vectors for genomic integration mediated by PiggyBac transposase. TF expression was controlled by a tetracycline-inducible promoter. Vectors encoded a puromycin-resistance cassette for the selection of cells with successful genomic integration. Each TF expression cassette encodes a unique DNA barcode, thus allowing the identification of TFs and the quantification of their frequency in cell pools.

Vectors were pooled in equimolar ratios to ensure even representation of TFs followed by retransformation in bacteria and large-scale DNA preparation. A mix of TF library and PiggyBac transposase was nucleofected into iPSCs. Cell culture scale and nucleofection parameters were optimized to ensure an adequate coverage of the high-plexity combinatorial space.

### Screening Outline

Three replicates of a prioritisation screening experiment were performed. Prior to nucleofection, iPSCs were expanded in GIBCO ESSENTIAL 8 Medium (Gibco) on standard tissue culture plates coated with Vitronectin (Life Technologies). Following nucleofection, cells were cultured as above and selected for successful genomic integration by adding puromycin to the culture media. Following selection, iPSCs were plated for reprogramming and cultured for up to 9 days in media promoting maintenance and survival of pancreatic beta cells, in the presence of doxycycline. At all stages, enough cells were maintained in culture to ensure adequate coverage for each combination of TFs. After reprogramming, cells were harvested and immunostained with antibodies targeting the pancreatic beta cells marker Insulin C-peptide, followed by fluorescence-activated cell sorting of marker-positive and negative control populations.

Bulk TF barcode quantification and analysis

Genomic DNA was extracted from all marker-positive and control samples and the relative distribution of TF barcodes in each bulk gDNA sample was quantified by amplicon sequencing.

Data were analysed using the Mageck method (Li et al. Genome Biol 15, 554, 2014). TF barcode counts were analysed using the Mageck count function. Subsequently, the raw counts files were used in both the test and maximum likelihood estimation (MLE) functions to generate coefficients and statistics on the enrichment of each TF in insulin C-peptide positive samples compared to a non-enriched control sample. The MLE function allows the comparison of multiple conditions and produces a beta score which is similar to the logFC value of a traditional differential expression test. This analysis considered reproducibility across the two replicates.

The test function compares conditions pairwise and produces a logFC value and associated statistics for each pair.

### Results:

The enrichment of TFs in the Insulin positive populations was measured by beta score and logFC as produced by the MLE and test functions, respectively. A TF was considered a putative reprogramming factor if beta score >=0.75 in Insulin C-peptide positive samples. NEUROG3 was also included as a putative reprogramming factor for further investigation. The top 5 TFs were selected for downstream consolidation (see Example 2 below).

TABLE 2

| Gene | Beta score |
|---|---|
| GLIS3 | 3.43 |
| NKX6-2 | 1.92 |
| PDX1 | 1.59 |
| NEUROD1 | 1.40 |
| ONECUT1 | 1.36 |
| BHLHE41 | 1.20 |
| TEAD1 | 1.17 |
| RFX2 | 1.16 |
| CTCFL | 1.01 |
| MYT1L | 0.96 |
| MAF | 0.92 |
| RARA | 0.85 |
| BHLHE 40 | 0.84 |
| MAFB | 0.84 |
| ASCL1 | 0.83 |
| BHLHA15 | 0.81 |
| FOXK1 | 0.77 |
| MNX1 | 0.77 |
| GTF2IRD1 | 0.75 |
| ISL1 | 0.75 |
| NEUROG3 | 0.60 |

Table 2 lists the 21 putative reprogramming TFs and indicates the beta score for Insulin positive samples. Beta scores include experimental replicates when calculated. Not shown are the TFs that failed the conditions for downstream validation.

### Example 2—Consolidation Screen

### Methods:

Following the prioritisation screen (see Example 1) different combinations of the 5 top scoring TFs (GLIS3, NKX6-2, PDX1, NEUROD1, ONECUT1) were assessed for reprogramming potential.

### Plasmid Library Encoding the TFs and Transposase-Mediated Delivery

Each of the TFs and negative controls were cloned separately into vectors for genomic integration mediated by PiggyBac transposase. TF expression was controlled by a tetracycline-inducible promoter. Vectors encoded a puromycin-resistance cassette for the selection of cells with successful genomic integration. Different combinations of vectors were pooled in equimolar ratios to ensure even representation of TFs. A mix of TF library and PiggyBac transposase was nucleofected into iPSCs. Cell culture scale and nucleofection parameters were optimized to ensure an adequate coverage of the high-plexity combinatorial space.

### Screening Outline

Two replicates of a consolidation screening experiment were performed using the same pooled plasmid libraries. To identify which TF was either necessary or sufficient to induce iPSC reprogramming to pancreatic beta cells, we compared the reprogramming efficiency of the full set against sets including all TFs except one (all-minus-one), and a single TF in the case of GLIS3. In total, 7 TF pools were assessed. To normalize TF dosage across the different pools, a negative control vector was added to the pool where necessary (Table 3).

TABLE 3
The composition of 7 TF pools assessed
in the consolidation screen.

| | Full set | All-minus-one | | | | | Single TF |
|---|---|---|---|---|---|---|---|
| Pool | 1 | 2 | 3 | 4 | 5 | 6 | 7 |
| GLIS3 | + | − | + | + | + | + | + |
| NKX6-2 | + | + | − | + | + | + | − |
| PDX1 | + | + | + | − | + | + | − |
| NEUROD1 | + | + | + | + | − | + | − |
| ONECUT1 | + | + | + | + | + | − | − |
| Neg | − | + | + | + | + | + | + |

Prior to nucleofection iPSCs were expanded in GIBCO ESSENTIAL 8 Medium (Gibco) on standard tissue culture plates coated with Vitronectin (Life Technologies). Following nucleofection, cells were cultured as above and selected for successful genomic integration by adding puromycin to the culture media. Following selection, iPSCs were plated for reprogramming and cultured for 8 days in media promoting maintenance and survival of pancreatic beta cells, in the presence of doxycycline. TF delivery by transposase may result in a variable number of insertion events, therefore biasing the analysis of reprogramming efficiency. Quantitative PCR was employed to verify that all pools were delivered with comparable efficiency.

After reprogramming, cells were harvested and processed for RNA extraction, and reverse transcription and quantitative PCR was performed for pancreatic beta cell markers Insulin and MAFA, and housekeeping controls. Alternatively, cells were fixed and immunostained for pancreatic beta cell markers insulin C-peptide and MAFA.

### Results:

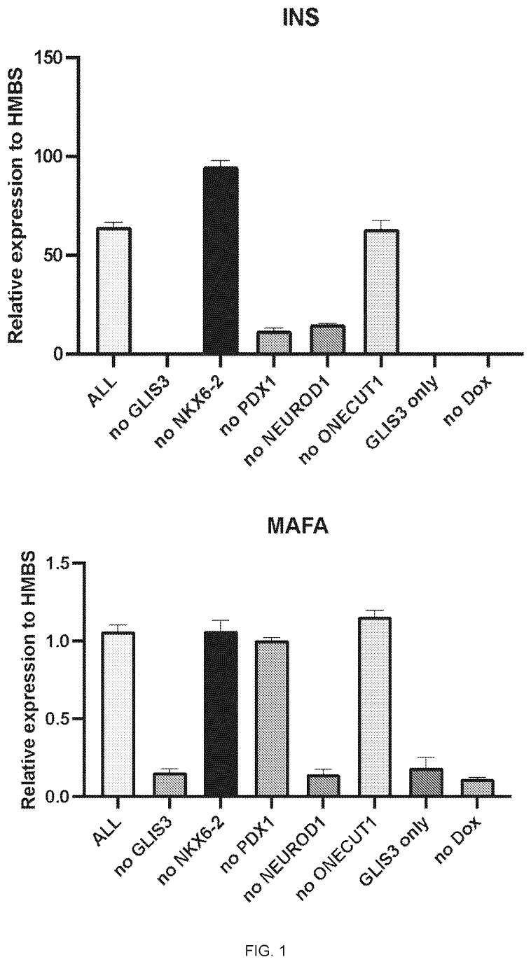

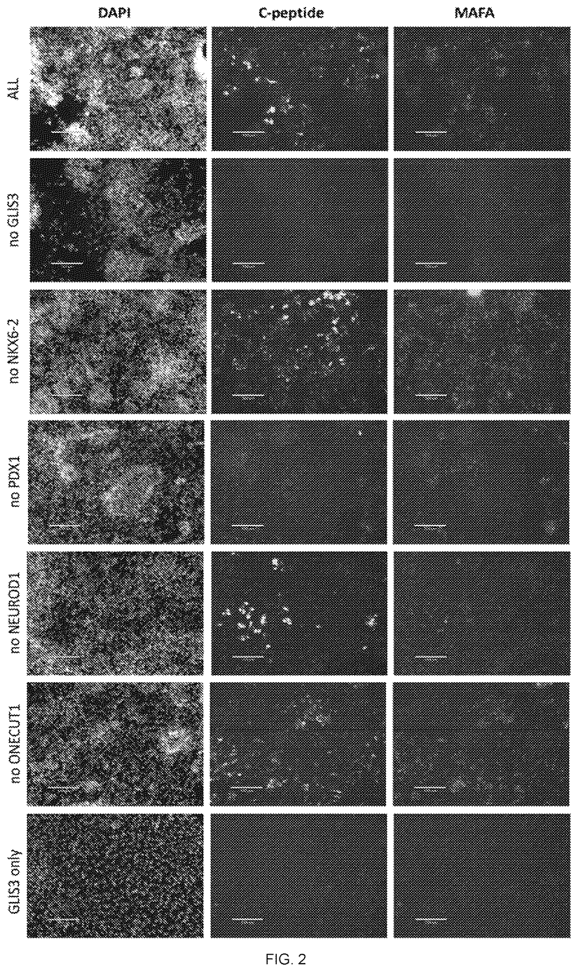

Reprogramming efficiency was assessed by measuring the expression level of the pancreatic beta cell markers Insulin (INS) and MAFA at the transcript level (FIG. 1), and insulin C-peptide and MAFA at the protein level (FIG. 2).

High insulin transcript levels were detectable in response to reprogramming by the full set of 5 TFs GLIS3, NKX6-2, PDX1, NEUROD1 and ONECUT1. Pools lacking GLIS3, PDX1 or NEUROD1 induced little or no INS transcript expression, indicating that they are likely to be necessary to reprogramming and/or for inducing insulin expression. Pools lacking either NKX6-2 or ONECUT1 insulin levels are less affected. The analysis of MAFA expression indicates GLIS3 and NEUROD1 as also likely to be necessary for reprogramming and/or for inducing MAFA expression (FIG. 1).

Similarly, high insulin C-peptide protein levels were detectable in response to reprogramming by the full set of 5 TFs GLIS3, NKX6-2, PDX1, NEUROD1 and ONECUT1. Pools lacking GLIS3 or PDX1 failed to induce INS protein expression, indicating that they are likely to be necessary for reprogramming and/or for inducing insulin expression. Pools lacking either NEUROD1, NKX6-2 or ONECUT1 insulin levels are less affected. The analysis of MAFA expression indicates GLIS3 and PDX1 as also likely to be necessary for reprogramming and/or for inducing MAFA expression (FIG. 2).

Taken together, these data indicate that GLIS3, PDX1 and NEUROD1 are key factors for the reprogramming of iPSCs to pancreatic beta cells.

### Example 3—Validation Screen

### Methods:

### Plasmid Library Encoding the TFs and Transposase—Mediated Delivery

Following Prioritisation screen (Example 1), the 21 TFs passing the threshold were assessed for reprogramming potential. Each of the TFs and negative controls were cloned separately into vectors for genomic integration mediated by PiggyBac transposase. TF expression was controlled by a tetracycline-inducible promoter. Vectors encoded a puromycin-resistance cassette for the selection of cells with successful genomic integration. Each TF expression cassette encodes a unique DNA barcode. We designed primers for the concurrent amplification of TF and cell barcodes in the Parse Evercode workflow, allowing us to assign TF combinations to single-cell transcriptome profiles.

Vectors were pooled in equimolar ratios to ensure even representation of TFs followed by retransformation in bacteria and large-scale DNA preparation. A mix of TF library and PiggyBac transposase was nucleofected into iPSCs. Cell culture scale and nucleofection parameters were optimised to ensure an adequate coverage of the high-plexity combinatorial space.

### Screening Outline

Three replicates of a validation screening experiment were performed using the same pooled plasmid library. Prior to nucleofection, iPSCs were expanded in GIBCO ESSENTIAL 8 Medium on standard tissue culture plates coated with Vitronectin (Life Technologies). Following nucleofection, cells were cultured as above and selected for successful genomic integration by adding puromycin to the culture media. Following selection, iPSCs were plated for reprogramming and cultured for up to 8 days in media promoting maintenance and survival of pancreatic beta cells, in presence of doxycycline. At all stages, enough cells were maintained in culture to ensure adequate coverage for each combination of TFs.

After reprogramming, cells were harvested and analysed by ScRNA-seq using Parse Biosciences Evercode.

### Single-Cell Transcriptome Analysis and TF Barcode Capture:

Non-sorted cells were purified and analysed by scRNA-seq using Parse Biosciences Evercode WT Mega Reagent Kits v2 following manufacturer's instructions for 1 million cells. Three of sixteen sub-libraries were then sequenced to obtain data for ~50,000 cells per replicate. After the cDNA amplification step, gene expression libraries were created and sequenced on a NovaSeq aiming for at least 25,000 reads per cell, as per the Parse Biosciences Evercode v2 protocol. In addition to the gene expression libraries, the matched cDNA was used as a template for further targeted amplification of the TF barcodes, which were sequenced on a NovaSeq.

### Data Analysis

Single-cell gene expression data was processed using the Parse split-pipe pipeline and TF barcode reads processed through a custom workflow. Parse cell barcodes were used to assign exogenous transcription factor (eTF) barcodes to single cells and their corresponding gene expression profiles. The data were further analysed in Seurat v4 (Stuart et al., Cell, 2019) and visualised on uniform manifold approximation and projection (UMAP) plots (Becht, Nature Biotech, 2019). Cell identities were assigned using insulin (INS) as a marker gene. Enrichment of eTFs either in single or in combinations was quantified by comparing eTF expression in marker-positive cells to marker-negative cells.

### Results:

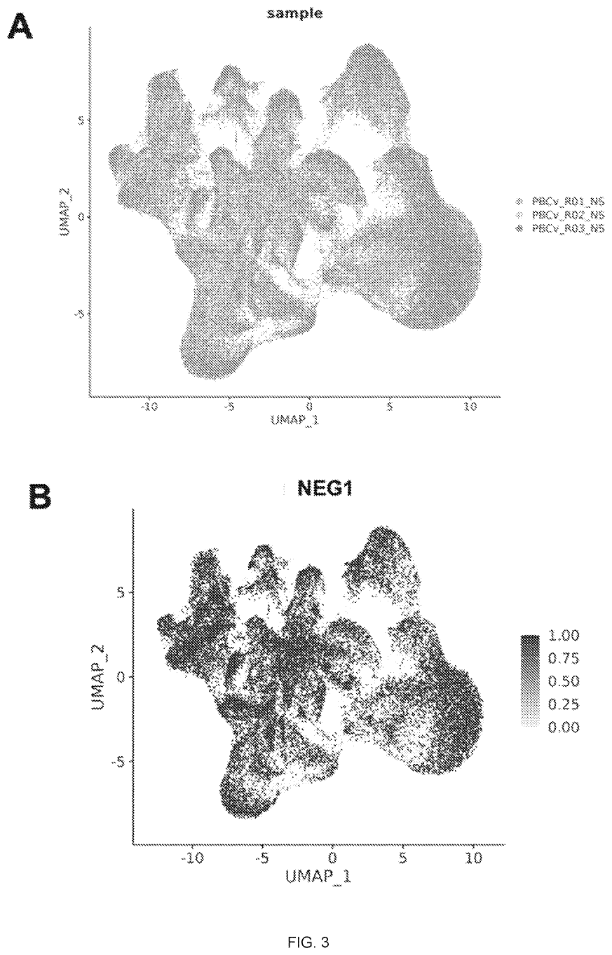

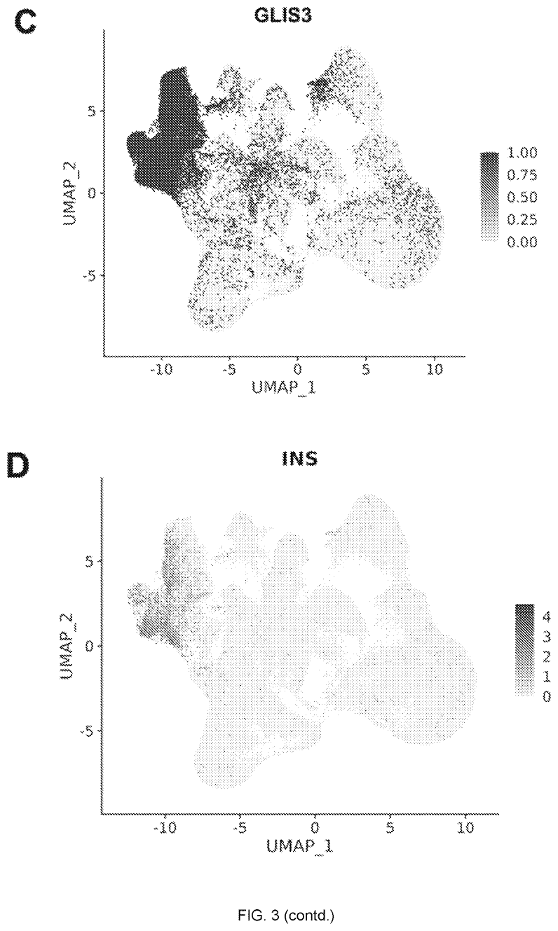

Single-cell gene expression data were visualised on UMAP plots including reprogrammed cells from all three replicates (FIG. 3A). There is a good overlap across the three replicates and different clusters are observed as expected from a reprogrammed heterogeneous population.

eTF barcodes were successfully detected by Parse Biosciences Evercode and assigned to single cells in all samples. While negative controls (e.g. NEG1 to NEG4) are distributed randomly across the samples (FIG. 3B), several eTFs are found in specific clusters within the cell population (e.g. GLIS3, FIG. 3C).

Next, cell identities were assigned using INS as a marker gene. A defined cluster of cells expressing these markers was identified (FIG. 3D).

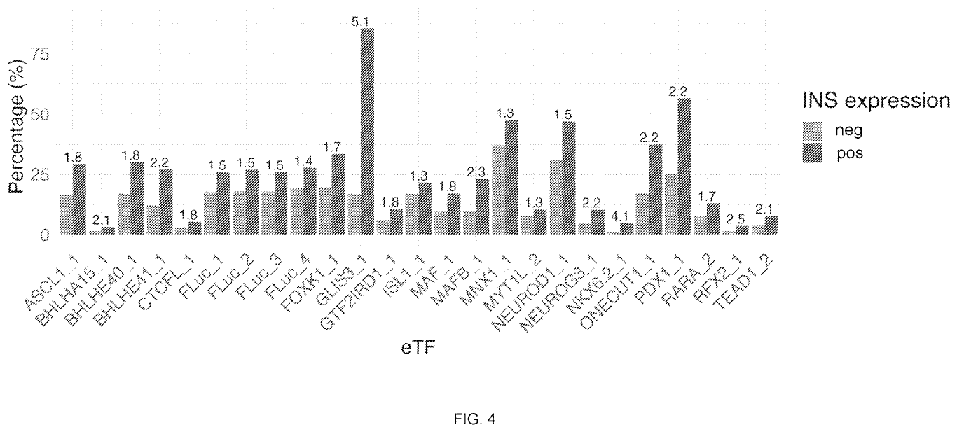

To identify eTFs reprogramming iPSCs to Pancreatic Beta cells, single eTF enrichment was quantified by comparing eTF expression in INS positive cells to markers-negative cells. Top enriched eTFs in INS cells are GLIS3, PDX1, NEUROD1, MNX1 and ONECUT1 (FIG. 4). To identify combinations of factors important for Pancreatic Beta cells reprogramming we looked at enriched combinations in insulin expressing cells vs negative cells. Combinations which showed a false discovery rate (FDR) below 10e-3 were considered statistically significant. In addition to the five factors above, FOXK1 and ASCL1 were present in high confidence combinations (Table 4 below). Taken together, these data indicate that GLIS3, PDX1, NEUROD1, MNX1, ONECUT1, FOXK1 and ASCL1 are important for reprogramming iPSCs into Pancreatic Beta cells.

TABLE 4

| | | % of INS positive |
|---|---|---|
| | FDR (All cells | cells with exact |
| eTF Combinations | with eTFs) | TF combination |
| GLIS3_NEUROD1_PDX1 | 4.19E−36 | 30.13 |
| GLIS3_PDX1 | 2.97E−26 | 11.34 |
| GLIS3_MNX1_PDX1 | 7.55E−25 | 30.77 |
| GLIS3_ONECUT1_PDX1 | 1.45E−23 | 31.25 |
| FOXK1_GLIS3_PDX1 | 1.06E−21 | 24.62 |
| ASCL1_GLIS3_PDX1 | 1.61E−06 | 31.03 |

### Example 4—Validation of TF Combination in a Controlled Transcription System Methods

To further validate TF combinations we have utilised the controlled transcription system as described in WO2018096343. In this system induced transgene over-expression is achieved by using the Tet-ON system components with transgene expression controlled by doxycycline.

The components are split between two genomic safe harbour sites (GSH) to reduce the risk of epigenetic gene silencing. The components are (i) transcriptional activator protein (reverse tetracycline trans-activator (rtTA)), which in the presence of doxycycline binds (ii) tetracycline response element (TRE; multiple TetO repeat sequences & minimal Cytomegalovirus (CMV) promoter).

An inducible transgene containing transcription factors was inserted into a genomic safe harbour. After electroporation of the Tet-ON system components, cells were cultured in GIBCO ESSENTIAL 8 Medium (Gibco) on standard tissue culture plates coated with Vitronectin (Life Technologies) and selected for successful genomic integration by adding puromycin to the culture media.

TABLE 5
List of polyclonal transcription factor combinations tested

| Line | Name | TFs |
|---|---|---|
| 1 | PG | PDX1 - GLIS3 |
| 2 | POG | PDX1 - ONECUT1 - GLIS3 |
| 3 | PNG | PDX1 - NEUROD1 - GLIS3 |

Following selection, polyclonal cell lines were plated for reprogramming and cultured for 8 days in media promoting maintenance and survival of pancreatic beta cells, in the presence of doxycycline to induce transgene expression. After reprogramming, cells were fixed and immunostained for pancreatic beta cell marker insulin C-peptide and for the transcription factor PDX1. Quantitative data was also generated using flow cytometry. Alternatively, cells were harvested and processed for RNA extraction, reverse transcription and quantitative PCR for pancreatic beta cell markers.

### Results:

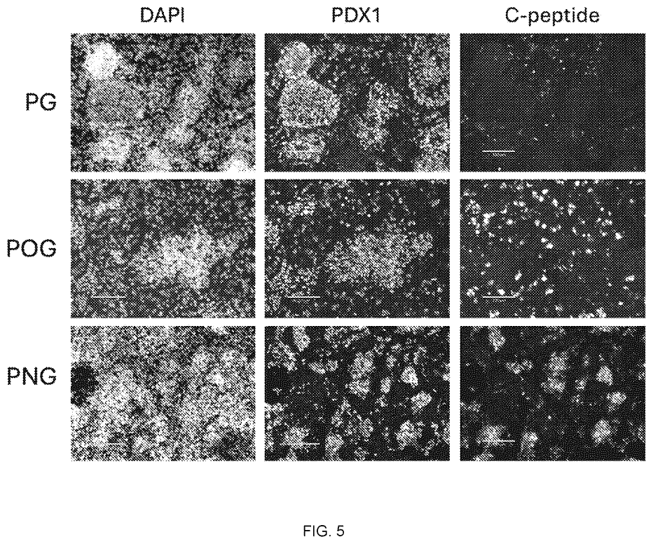

To assess reprogramming efficiency, immunocytochemistry (ICC) for the TF PDX1 (present in all transgene cassettes) and for the pancreatic beta cell marker insulin C-peptide was performed. Protein expression of PDX1 was readily detectable in all cell lines tested. Expression of insulin C-peptide was detected in the PG line but is highly present in POG and PNG lines (FIG. 5). 8.8% of PDX1-expressing cells were also expressing insulin C-peptide in PG. This number increases to 38.5% for POG and to 79% for PNG, as analysed by flow cytometry.

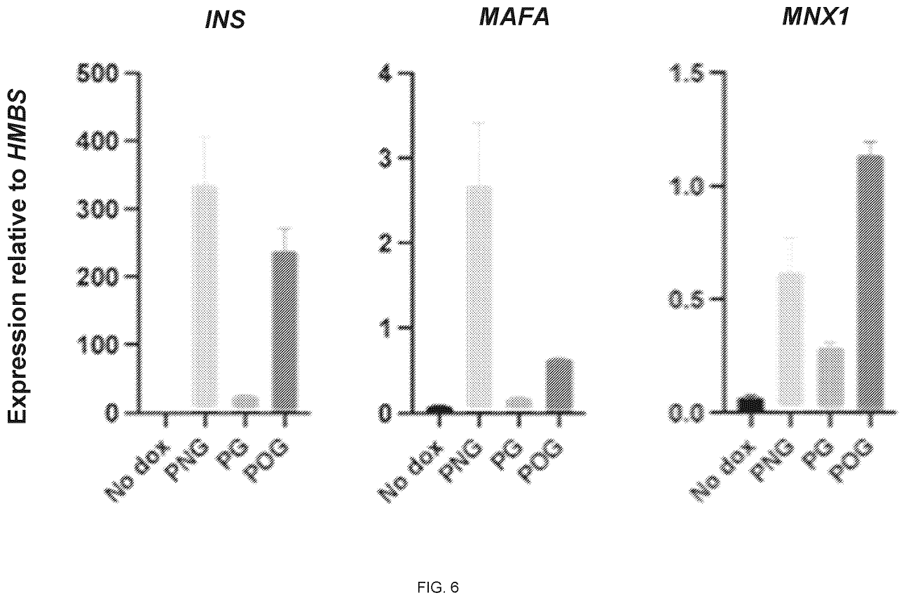

Similarly, transcript level analysis showed that expression of key beta cells markers INS, MAFA and MNX1 are enriched in the PG, POG and PNG lines but not in the negative control lacking doxycycline (No dox) (FIG. 6). The highest expression levels were seen in the POG and PNG lines.

1. A method of generating pancreatic beta cells comprising expressing one or more polypeptides having the activity of one or more transcription factors and/or increasing the expression of one or more transcription factors, the transcription factors selected from the group consisting of: GLIS3, PDX1, NEUROD1, NKX6-2, ONECUT1 and variants thereof, in a cell population and culturing the cell population to obtain pancreatic beta cells.

2. The method as defined in claim 1, wherein one or more of the transcription factors comprises GLIS3, PDX1 or NEUROD1.

3. The method as defined in claim 1 or claim 2, wherein the transcription factors comprise PDX1 and GLIS3.

4. The method as defined in any one of claims 1 to 3, which comprises increasing the expression of one or more additional transcription factors as listed in Table 1.

5. The method as defined in claim 3 or claim 4, wherein the transcription factors comprise PDX1, GLIS3 and one or more additional transcription factors selected from the list consisting of: (1) NEUROD1, (2) ONECUT1, (3) MNX1, (4) FOXK1, (5) ASCL1, preferably NEUROD1, more preferably NEUROD1 in combination with FOXK1 and/or ONECUT1.

6. The method as defined in any one of claims 1 to 5, wherein the method comprises increasing the expression of between three and seven transcription factors, in particular five transcription factors.

7. The method as defined in any one of claims 1 to 6, wherein the cell population comprises pluripotent stem cells, in particular induced pluripotent stem cells.

8. The method as defined in any one of claims 1 to 7, which additionally comprises monitoring the cell population for at least one characteristic of a pancreatic beta cell.

9. The method as defined in claim 8, wherein the characteristic is selected from one or more of:
(i) expression of one or more cell markers, such as proinsulin, MAFA or a combination thereof;
(ii) secretion of insulin or amylin; and
(iii) pancreatic beta cell morphological features.

10. The method as defined in any one of claims 1 to 9, wherein the expression of the transcription factors is increased by contacting the cell population with one or more exogenous expression cassettes encoding one or more of the genes, or one or more agents that activate or increase the expression or amount of the transcription factors.

11. The method as defined in any one of claims 1 to 10, wherein expression of the genes is under controlled transcription.

12. The method as defined in any one of claims 1 to 11, wherein a sequence encoding one or more of the transcription factors is introduced into the cell population using a method comprising:
insertion of a coding sequence for a transcriptional regulator protein into a first genomic safe harbour site of a source cell present in the cell population; and
insertion of an inducible cassette into a second genomic safe harbour site of the source cell, wherein said inducible cassette comprises said sequence encoding one or more transcription factors operably linked to an inducible promoter, and said promoter is regulated by the transcriptional regulator protein.

13. The method as defined in any one of claims 1 to 12, which comprises:
(i) culturing under suitable conditions for at least 3 days, such as at least 7 days, in particular about 8 days, and/or
(ii) culturing in media comprising one or more components selected from the group consisting of: Heparin and Bovine Serum Albumin without fatty acids.

14. A method for the production of pancreatic beta cells from a source cell, comprising the steps of:
a) insertion of a gene encoding a transcriptional regulator protein into a first genomic safe harbour site of the source cell; and
b) insertion of at least one nucleotide sequence encoding one or more polypeptides having the activity of one or more transcription factors and/or encoding one or more transcription factors, the transcription factors selected from the group consisting of: GLIS3, PDX1, NEUROD1, NKX6-2, ONECUT1 and variants thereof, operably linked to an inducible promoter into one or more further genomic safe harbour sites of the source cell, wherein said inducible promoter is regulated by the transcriptional regulator protein; and
c) culturing the source cell(s) comprising the insertions to obtain pancreatic beta cells.

15. A cell comprising one or more exogenous expression cassettes comprising nucleotide sequences encoding one or more polypeptides having the activity of one or more transcription factors and/or encoding one or more transcription factors, wherein the one or more transcription factors are selected from the group consisting of: GLIS3, PDX1, NEUROD1, NKX6-2, ONECUT1 and variants thereof.

16. A cell as defined in claim 15, for use in therapy, in vitro diagnostics or drug screening.

17. A kit for differentiating a cell into a pancreatic beta cell comprising:
(i) a source cell and an agent that activates or increases the expression or amount of one or more transcription factors; and/or
(ii) one or more expression cassette(s) comprising nucleotide sequences encoding one or more polypeptides having the activity of one or more transcription factors and/or encoding one or more transcription factors,
wherein the one or more transcription factors selected from the group consisting of: GLIS3, PDX1, NEUROD1, NKX6-2, ONECUT1 and variants thereof.
